(19) United States
(12) Patent Application Publication
(10) Pub. No.: US 2006/0228413 A1
(43) Pub. Date: Oct. 12, 2006

(54) Controlled release venlafaxine formulations

(75) Inventors: Sara Ketsela, Danbury, CT (US); Dean Dinicola, Warren, CT (US); Anand R. Baichwal, Wappingers Falls, NY (US)

Correspondence Address: DAVIDSON, DAVIDSON & KAPPEL, LLC, 485 SEVENTH AVENUE, 14TH FLOOR, NEW YORK, NY, 10018, US

(73) Assignee: Penwest Pharmaceuticals Co., Patterson, NY (US)

(21) Appl. No.: 11/363,845

(22) Filed: Feb. 28, 2006

Related U.S. Application Data
(60) Provisional application No. 60/657,035, filed on Feb. 28, 2005.
(60) Provisional application No. 60/750,594, filed on Dec. 14, 2005.

(51) Int. Cl. A61K 9/22

(52) U.S. Cl. 424/468

(57) ABSTRACT

In certain embodiments, the present invention is directed to a controlled release oral dosage form comprising: a therapeutically effective amount of venlafaxine or a pharmaceutically acceptable salt thereof and a controlled release material; wherein the amount of venlafaxine or pharmaceutically acceptable salt thereof released at 1 hour in 900 mL 0.1 N HCl (pH 1.5) with 40% EtOH using USP Apparatus II at 50 rpm is within 25% of the amount of venlafaxine or pharmaceutically acceptable salt thereof released at 1 hour in 900 mL 0.1 N HCl (pH 1.5) using USP Apparatus II at 50 rpm.

This application claims priority from U.S. Provisional Application No. 60/657,035, filed Feb. 28, 2005, and U.S. Provisional Application No. 60/750,594, filed Dec. 14, 2005, the disclosures of which are hereby incorporated by reference in their entireties.

### FIELD OF THE INVENTION

The present invention relates to controlled release oral tablets containing a therapeutically effective amount of venlafaxine, an active metabolite of venlafaxine, or a pharmaceutically acceptable salt thereof. The present invention is further related to methods of preparing such formulations, and to methods of treatment utilizing such formulations. The present invention further relates to controlled release dosage forms containing a therapeutically effective amount of venlafaxine, an active metabolite of venlafaxine, or a pharmaceutically acceptable salt thereof which are resistant to alcohol induced dose dumping.

### BACKGROUND OF THE INVENTION

The advantages of controlled release products are well known in the pharmaceutical field and include the ability to maintain a desired blood level of a medicament over a comparatively longer period of time while increasing patient compliance by reducing the number of administrations necessary to achieve the same. These advantages have been attained by a wide variety of methods.

For example, different hydrogels have been described for use in controlled release medicines, some of which are synthetic, but most of which are semi-synthetic or of natural origin. A few contain both synthetic and non-synthetic material. However, some of the systems require special process and production equipment, and in addition some of these systems are susceptible to variable drug release.

Oral controlled release delivery systems should ideally be adaptable so that release rates and profiles can be matched to physiological and chronotherapeutic requirements.

While many controlled and sustained-release formulations are already known, certain soluble to highly soluble drugs present formulation difficulties when included in such formulation. An example of such a highly soluble drug is venlafaxine hydrochloride.

Currently, venlafaxine is available as 150mg, 75mg, and 37.5mg extended release capsules and is marketed under the name Effexor XR® by Wyeth-Ayerst Company.

Effexor XR® is susceptible to alcohol induced dose dumping. Accordingly, if the dosage form is administered with an amount of alcohol, the integrity of the controlled release mechanism of the dosage form will be compromised and a potentially toxic amount of venlafaxine may be available for immediate release.

U.S. Pat. No. 6,274,171 to Sherman et al. reports that numerous attempts to produce venlafaxine extended release tablets by hydrogel technology proved to be fruitless because the compressed tablets were either physically unstable (poor compressibility or capping problems) or dissolved too rapidly in dissolution studies.

Accordingly, there exists a need in the art to provide a controlled release oral tablet that provides for the extended release of venlafaxine (e.g., venlafaxine hydrochloride) suitable for once-a-day administration utilizing hydrogel technology.

There also exists a need in the art to provide a controlled release dosage form that provides for the extended release of venlafaxine (e.g., venlafaxine hydrochloride) which has reduced potential for alcohol induced dose dumping.

All documents cited herein, including the foregoing, are incorporated by reference in their entireties for all purposes.

### OBJECTS AND SUMMARY OF THE INVENTION

It is an object of the present invention to provide an oral controlled release formulation comprising venlafaxine, an active metabolite of venlafaxine or a pharmaceutically acceptable salt thereof suitable for twice-a-day or once-a-day administration.

It is a further object of certain embodiments of the present invention to provide an oral controlled release dosage form which releases venlafaxine, an active metabolite of venlafaxine or a pharmaceutically acceptable salt thereof over an extended period of time, e.g., for a time period of at least about 24 hours, when the dosage form is exposed to an environmental fluid (e.g., the gastrointestinal tract).

It is a further object of certain embodiments of the present invention to provide a controlled release oral dosage form which is bioequivalent to a commercialized extended release dosage form comprising venlafaxine hydrochloride (e.g., Effexor XR).

It is a further object of certain embodiments of the present invention to provide methods for preparing the controlled release oral dosage forms disclosed herein.

It is a further object of certain embodiments of the present invention to provide a method of treating depression comprising administering the controlled release oral dosage forms disclosed herein.

It is a further object of certain embodiments of the present invention to provide a method for reducing the level of nausea and/or incidence of emesis associated with multiple daily dosing of venlafaxine, active metabolite of venlafaxine or salt thereof, which comprises dosing a patient in need of treatment with venlafaxine, the controlled release oral dosage forms disclosed herein.

It is a further object of certain embodiments of the present invention to provide an oral controlled release formulation comprising venlafaxine, an active metabolite of venlafaxine or a pharmaceutically acceptable salt thereof which has reduced potential for alcohol induced dose dumping.

It is an object of certain embodiments of the present invention to provide an oral controlled release formulation comprising venlafaxine, an active metabolite of venlafaxine or a pharmaceutically acceptable salt thereof which has reduced potential for alcohol induced dose dumping and is suitable for once-a-day administration.

It is a further object of certain embodiments of the present invention to provide an oral controlled release dosage form which has reduced potential for alcohol induced dose dumping and which releases venlafaxine, an active metabolite of venlafaxine or pharmaceutically acceptable salt thereof over an extended period of time, e.g., for a time period of at least about 24 hours, when the tablets are exposed to an environmental fluid (e.g., the gastrointestinal tract).

It is a further object of certain embodiments of the present invention to provide a controlled release oral dosage form which has reduced potential for alcohol induced dose dumping as compared to a commercialized extended release dosage form comprising venlafaxine hydrochloride (e.g., Effexor XR).

It is a further object of certain embodiments of the present invention to provide a controlled release oral dosage form which has reduced potential for alcohol induced dose dumping and which is bioequivalent to a commercialized extended release dosage form comprising venlafaxine hydrochloride (e.g., Effexor XR).

It is a further object of certain embodiments of the present invention to provide a controlled release oral dosage form which has reduced potential for alcohol induced dose dumping as compared to the extended release dosage form comprising venlafaxine hydrochloride approved by the FDA under NDA application No. 020699 on Oct. 20, 1997.

It is a further object of certain embodiments of the present invention to provide a controlled release oral dosage form which has reduced potential for alcohol induced dose dumping and which is bioequivalent to the extended release dosage form comprising venlafaxine hydrochloride approved by the FDA under NDA application No. 020699 on Oct. 20, 1997.

It is a further object of certain embodiments of the present invention to provide a method of reducing alcohol induced dose dumping of venlafaxine, an active metabolite of venlafaxine, or pharmaceutically acceptable salt thereof, from a controlled release dosage form comprising preparing a controlled release dosage form disclosed herein.

The above-mentioned objects and others are achieved by virtue of the present invention, which is directed in part to a controlled release oral solid dosage form comprising a matrix comprising a therapeutically effective amount of venlafaxine, an active metabolite of venlafaxine, or a pharmaceutically acceptable salt thereof, dispersed in a cross-linked gelling agent, the matrix providing a controlled release of venlafaxine, active metabolite of venlafaxine, or salt thereof to provide 24 hour therapeutic plasma levels after oral administration to human patients. p In certain embodiments, the present invention is directed to a controlled release oral solid dosage form comprising a matrix comprising a therapeutically effective amount of venlafaxine, an active metabolite of venlafaxine, or a pharmaceutically acceptable salt thereof, dispersed in a gelling agent; the gelling agent comprising a heteropolysaccharide gum and a homopolysaccharide gum; and the matrix providing a controlled release of venlafaxine, active metabolite of venlafaxine, or salt thereof to provide 24 hour therapeutic plasma levels after oral administration to human patients.

In certain embodiments, the present invention is directed to a controlled release oral solid dosage form comprising a matrix comprising a therapeutically effective amount of venlafaxine, an active metabolite of venlafaxine, or a pharmaceutically acceptable salt thereof, dispersed in a cross-linked gelling agent comprising a heteropolysaccharide gum and an effective amount of an ionizable gel strength enhancing agent, the dosage form providing an in-vitro dissolution rate, when tested in USP Apparatus Type III at 37° C.±0.5 in 250 ml (per dissolution vessel) at 15 dpm at a pH of 1.5 for the first hour, with a switch to pH 4.5 for two hours, with a switch to pH 7.5 thereafter, of from about 10% to about 50% venlafaxine, active metabolite or salt thereof released at 2 hours; from about 30% to about 65% venlafaxine, active metabolite or salt thereof released at 4 hours; from about 40% to about 80% venlafaxine, active metabolite or salt thereof released at 8 hours; and less than about 95% venlafaxine, active metabolite or salt thereof released at about 16 hours.

In certain embodiments, the present invention is directed to a controlled release oral solid dosage form comprising a matrix comprising a therapeutically effective amount of venlafaxine, an active metabolite of venlafaxine, or a pharmaceutically acceptable salt thereof, dispersed in a gelling agent; the matrix further comprising a hydrophobic material, the matrix providing a controlled release of venlafaxine, active metabolite of venlafaxine, or salt thereof to provide 24 hour therapeutic plasma levels after oral administration to human patients.

In certain embodiments, the present invention is directed to a controlled release oral solid dosage form comprising a matrix comprising a therapeutically effective amount of venlafaxine, an active metabolite of venlafaxine, or a pharmaceutically acceptable salt thereof, dispersed in a gelling agent; the matrix providing an vitro dissolution rate, when measured by the USP Apparatus Type III at 37° C.±0.5 in 250 ml at 15 dpm at a pH of 1.5 for the first hour, with a switch to pH 4.5 thereafter, of greater than 30% venlafaxine or salt thereof released at 2 hours; the matrix providing a controlled release of the active agent to provide 24 hour therapeutic plasma levels after oral administration to human patients.

In certain embodiments, the present invention is directed to a controlled release oral dosage form comprising a matrix comprising therapeutically effective amount of venlafaxine, an active metabolite of venlafaxine or a pharmaceutically acceptable salt thereof dispersed in a gelling agent; the matrix providing a controlled release of venlafaxine, active metabolite or salt thereof to provide 24 hour therapeutic plasma levels of after oral administration to human patients, wherein the dosage form does not comprise sodiumcarboxymethylcellulose.

A controlled release oral solid dosage form comprising a therapeutically effective amount of venlafaxine, an active metabolite of venlafaxine, or a pharmaceutically acceptable salt thereof, and a controlled release material; wherein the amount of venlafaxine, active metabolite of venlafaxine, or pharmaceutically acceptable salt thereof released at I hour in 900 mL 0.1 N HCl (pH 1.5) with 40% EtOH using USP Apparatus II at 50 rpm is within 25% of the amount of venlafaxine, active metabolite of venlafaxine, or pharmaceutically acceptable salt thereof released at 1 hour in 900 mL 0.1 N HCl (pH 1.5) using USP Apparatus II at 50 rpm.

A controlled release oral solid dosage form comprising a therapeutically effective amount of venlafaxine, an active metabolite of venlafaxine, or a pharmaceutically acceptable salt thereof, and a controlled release material; wherein the amount of venlafaxine, active metabolite of venlafaxine, or pharmaceutically acceptable salt thereof released at 1 hour in 900 mL 0.1 N HCl (pH 1.5) with 40% EtOH using USP Apparatus II at 50 rpm is less than the amount of venlafaxine, active metabolite of venlafaxine, or pharmaceutically acceptable salt thereof released at 1 hour in 900 mL 0.1 N HCl (pH 1.5) using USP Apparatus 11 at 50 rpm.

In certain embodiments, the present invention is directed to a controlled release oral dosage form comprising a matrix comprising a therapeutically effective amount of venlafaxine, active metabolite of venlafaxine or a pharmaceutically acceptable salt thereof dispersed in a gelling agent, the gelling agent comprising a microbial polysaccharide gum; and the matrix providing a controlled release of venlafaxine to provide 24 hour therapeutic plasma levels after oral administration to human patients.

In certain embodiments, the present invention is directed to a controlled release oral solid dosage form comprising a matrix comprising a therapeutically effective amount of venlafaxine, an active metabolite of venlafaxine or a pharmaceutically acceptable salt thereof, dispersed in s sustained release excipient; the sustained release excipient comprising from 60% to about 95% by weight of a gelling agent; from about 10% to about 30% by weight of an inert diluent; and from about 5 to about 15% by weight of an ionizable gel strength enhancing agent.

In certain embodiments, the present invention is directed to a controlled release oral solid dosage form comprising a matrix comprising a therapeutically effective amount of venlafaxine, an active metabolite of venlafaxine or a pharmaceutically acceptable salt thereof, dispersed in a sustained release excipient; the sustained release excipient comprising from 60% to about 95% by weight of a gelling agent; from about 10% to about 30% by weight of an inert diluent; and from about 5 to about 15% by weight of a hydrophobic material.

In certain embodiments, the present invention is directed to any of the controlled release dosage forms disclosed herein which provide therapeutic plasma levels for about 12 to about 30 hours after oral administration to human patients.

In certain embodiments, the present invention is directed to a controlled release oral dosage form comprising a matrix comprising (i) venlafaxine, an active metabolite of venlafaxine or a pharmaceutically acceptable salt thereof and (ii) at least one controlled release excipient; the dosage form being scored in order to divide the dosage form into at least two substantially equal divided doses.

In certain embodiments, the present invention is directed to a method of titrating a patient in need of venlafaxine therapy comprising: a) dividing a dosage form as disclosed herein into divided doses; b) administering a divided dose for at least one dosing interval to the patient; and c) increasing the dosage in a subsequent administration.

In certain embodiments, the present invention is directed to a method of titrating a patient in need of venlafaxine therapy comprising: a) dividing a dosage form as disclosed herein into divided doses; and b) administering to a patient currently on venlafaxine therapy a divided dose for at least one dosing interval in order to decrease the dosage to the patient. [0042] In certain embodiments of the present invention disclosed herein, the controlled release oral dosage form provides an in-vitro dissolution rate, when measured by the USP Apparatus Type III at 37° C.±0.5 in 250 ml (per dissolution vessel) at 15 dpm at a pH of 1.5 for the first hour, with a switch to pH 4.5 for two hours, with a switch to pH 7.5 thereafter, of from about 10% to about 50% venlafaxine, active metabolite or salt thereof released at 2 hours; from about 30% to about 65% venlafaxine, active metabolite or salt thereof released at 4 hours; from about 40% to about 80% venlafaxine, active metabolite or salt thereof released at 8 hours; and less than 100%, less than about 98% or less than about 95% venlafaxine, active metabolite or salt thereof released at 16 hours.

In certain embodiments of the present invention disclosed herein, the controlled release dosage form provides an in-vitro dissolution rate, when measured by the USP Apparatus Type III at 37° C.±0.5 in 250 ml (per dissolution vessel) at 15 dpm at a pH of 1.5 for the first hour, with a switch to pH 4.5 for two hours, with a switch to pH 7.5 thereafter of from about 10% to about 35% venlafaxine, active metabolite or salt thereof released at 2 hours; from about 30% to about 55% venlafaxine, active metabolite or salt thereof released at 4 hours; from about 50% to about 80% venlafaxine, active metabolite or salt thereof released at 8 hours; and less than 100%, less than about 98% or less than about 95% venlafaxine, active metabolite, or salt thereof released at 16 hours.

In certain embodiments of the present invention disclosed herein, the controlled release dosage form provides an in-vitro dissolution rate, when measured by the USP Apparatus Type III at 37° C.±0.5 in 250 ml (per dissolution vessel) at 15 dpm at a pH of 1.5 for the first hour, with a switch to pH 4.5 thereafter of from about 10% to about 35% venlafaxine, active metabolite thereof or salt thereof released at 2 hours.

In certain embodiments of the present invention disclosed herein, the controlled release dosage form provides an in-vitro dissolution rate, when measured by the USP Apparatus Type III at 37° C.±0.5 in 250 ml (per dissolution vessel) at 15 dpm at a pH of 1.5 for the first hour, with a switch to pH 4.5 for two hours, with a switch to pH 7.5 thereafter of from about 30% to about 55% venlafaxine, active metabolite thereof or salt thereof released at 4 hours.

In certain embodiments of the present invention disclosed herein, the controlled release dosage form provides an in-vitro dissolution rate, when measured by the USP Apparatus Type III at 37° C.±0.5 in 250 ml (per dissolution vessel) at 15 dpm at a pH of 1.5 for the first hour, with a switch to pH 4.5 for two hours, with a switch to pH 7.5 thereafter of from about 40% to about 80% venlafaxine, active metabolite thereof or salt thereof released at 8 hours.

In certain embodiments of the present invention disclosed herein, the controlled release dosage form provides a mean Cmax from about 120 ng/ml to about 180 ng/ml based on a 150 mg dose of venlafaxine.

In certain embodiments of the present invention disclosed herein, the controlled release dosage form provides a mean Tmax of from about 4 hours to about 8 hours after administration to a human patient.

In certain embodiments, the present invention is directed to a controlled release oral dosage form as disclosed herein which provides a mean Tmax of venlafaxine from about 4.4 hours to about 6.9 hours after single dose administration.

In certain embodiments, the present invention is directed to a controlled release oral dosage form as disclosed herein which provides a mean Tmax of venlafaxine of about 5.5 hours after single dose administration.

In certain embodiments, the present invention is directed to a controlled release oral dosage form as disclosed herein which provides a mean Tmax of O-desmethyl-venlafaxine from about 6 hours to about 12 hours after single dose administration.

In certain embodiments, the present invention is directed to a controlled release oral dosage form as disclosed herein which provides a mean Tmax of 0-desmethyl-venlafaxine from about 7.2 hours to about 11.25 hours after single dose administration.

In certain embodiments, the present invention is directed to a controlled release oral dosage form as disclosed herein which provides a mean Tmax of venlafaxine of about 9 hours after single dose administration.

In certain embodiments, the present invention is directed to a controlled release oral dosage form as disclosed herein which provides a mean Cmax of venlafaxine from about 100 ng/mL to about 200 ng/mL, based on administration of an amount equivalent to about 150 mg venlafaxine base.

In certain embodiments, the present invention is directed to a controlled release oral dosage form as disclosed herein which provides a mean Cmax of venlafaxine from about 120 ng/mL to about 188 ng/mL, based on administration of an amount equivalent to about 150 mg venlafaxine base.

In certain embodiments, the present invention is directed to a controlled release oral dosage form as disclosed herein which provides a mean Cmax of venlafaxine of about 150 ng/mL, based on administration of an amount equivalent to about 150 mg venlafaxine base.

In certain embodiments, the present invention is directed to a controlled release oral dosage form as disclosed herein which provides a mean Cmax of 0-desmethyl-venlafaxine from about 200 ng/mL to about 350 ng/mL, based on administration of an amount equivalent to about 150 mg venlafaxine base.

In certain embodiments, the present invention is directed to a controlled release oral dosage form as disclosed herein which provides a mean Cmax of O-desmethyl-venlafaxine from about 208 ng/mL to about 325 ng/mL, based on administration of an amount equivalent to about 150 mg venlafaxine base.

In certain embodiments, the present invention is directed to a controlled release oral dosage form as disclosed herein which provides a mean Cmax of O-desmethyl-venlafaxine of about 260 ng/mL, based on administration of an amount equivalent to about 150 mg venlafaxine base.

In certain embodiments, the present invention is directed to a controlled release oral dosage form as disclosed herein which provides a 24 hour therapeutic plasma levels of venlafaxine after oral administration to human patients.

The pharmacokinetic parameters for O-desmethyl-venlafaxine disclosed herein can be obtained by formulations comprising venlafaxine or a pharmaceutically acceptable salt thereof.

In certain embodiments of the present invention disclosed herein, the dissolution rate in-vitro of the dosage form, when measured by the USP Apparatus Type III at 37° C.±0.5 in 250 ml (per dissolution vessel) at 15 dpm, a pH of 1.5 for the first hour, with a switch to pH 4.5 for two hours, with a switch to pH 7.5 thereafter, is as follows: from 0% to about 50% venlafaxine, active metabolite or salt thereof released at about 1 hour; from about 30% to about 60% venlafaxine, active metabolite or salt thereof released at about 2 hours; from about 45% to about 80% venlafaxine, active metabolite or salt thereof released at about 4 hours; from about 60% to about 95% venlafaxine, active metabolite or salt thereof released at about 8 hours; greater than about 50% venlafaxine, active metabolite or salt thereof released at about 16 hours; greater than about 80% venlafaxine, active metabolite or salt thereof released at about 24 hours.

In certain embodiments of the present invention disclosed herein, the dissolution rate in-vitro of the dosage form, when measured by the USP Apparatus Type III at 37° C.±0.5 in 250 ml (per dissolution vessel) at 15 dpm, a pH of 1.5 for the first hour, with a switch to pH 4.5 for two hours, with a switch to pH 7.5 thereafter, is as follows: from about 15% to about 30% venlafaxine, active metabolite or salt thereof released at about 1 hour; from about 20% to about 40% venlafaxine, active metabolite or salt thereof released at about 2 hours; from about 30% to about 55% venlafaxine, active metabolite or salt thereof released at about 4 hours; from about 50% to about 75% venlafaxine, active metabolite or salt thereof released at about 8 hours; from about 80% to about 96% venlafaxine, active metabolite or salt thereof released at about 16 hours; greater than about 80% venlafaxine, active metabolite or salt thereof released at about 24 hours.

In certain embodiments of the present invention disclosed herein, the dissolution rate in-vitro of the dosage form, when measured by the USP Apparatus Type III at 37° C±0.5 in 250 ml (per dissolution vessel) at 15 dpm, a pH of 1.5 for the first hour, with a switch to pH 4.5 for two hours, with a switch to pH 7.5 thereafter, is as follows: from about 20% to about 30% venlafaxine, active metabolite or salt thereof released at about 1 hour; from about 30% to about 40% venlafaxine, active metabolite or salt thereof released at about 2 hours; from about 40% to about 50% venlafaxine, active metabolite or salt thereof released at about 4 hours; from about 60% to about 75% venlafaxine, active metabolite or salt thereof released at about 8 hours; from about 80% to about 96% venlafaxine, active metabolite or salt thereof released at about 16 hours; greater than about 80% venlafaxine, active metabolite or salt thereof released at about 24 hours.

In certain embodiments, the present invention is directed to a controlled release oral dosage form (e.g., tablet) comprising an effective amount of venlafaxine, or pharmaceutically acceptable salt thereof; and a gelling agent comprising a heteropolysaccharide gum and a homopolysaccharide gum capable of cross-linking the heteropolysaccharide gum when exposed to an environmental fluid, said dosage form providing a mean Cₘₐₓ from about 120 ng/ml to about 180 ng/ml based on administration of 150 mg venlafaxine; the dosage form providing a therapeutic effect for about 24 hours after oral administration, and/or a mean Tₘₐₓ 4 hours to about 8 hours after oral administration of the dosage form.

In certain embodiments, the present invention is directed to a controlled release oral dosage form (e.g., tablet) comprising an effective amount of venlafaxine or pharmaceutically acceptable salt thereof; a gelling agent comprising a heteropolysaccharide gum and a homopolysaccharide gum capable of cross-linking the heteropolysaccharide gum when exposed to an environmental fluid; optionally a cellulose derivative such as, e.g., an alkylcellulose, hydroxyalkylcellulose, hydroxypropylalkylcellulose, or mixtures thereof; optionally an inert diluent selected from, e.g., a monosaccharide, a disaccharide, a polyhydric alcohol, or mixtures thereof; and optionally an effective amount of a pharmaceutically acceptable water-soluble cationic cross-linking agent; said dosage form providing a mean Cmax from about 120 ng/ml to about 180 ng/ml based on administration of 150 mg venlafaxine; the dosage form providing a therapeutic effect for about 24 hours after oral administration and/or a mean Tₘₐₓ 4 hours to about 8 hours after oral administration of the dosage form.

In certain embodiments, the present invention is directed to a controlled release oral dosage form comprising a therapeutically effective amount of venlafaxine, an active metabolite of venlafaxine or a pharmaceutically acceptable salt thereof and a controlled release material; wherein the amount of venlafaxine, active metabolite or salt thereof released at 1 hour in 900 mL 0.1 N HCl (pH 1.5) with 40% EtOH using USP Apparatus II at 50 rpm is within 25%, within 15%, within 10%, or within 5% of the amount of venlafaxine, active metabolite or salt thereof released at I hour in 900 mL 0.1 N HCl (pH 1.5) using USP Apparatus II at 50 rpm.

In certain embodiments, the present invention is directed to a controlled release oral dosage form comprising: a therapeutically effective amount of venlafaxine, active metabolite of venlafaxine or a pharmaceutically acceptable salt thereof and a controlled release material; wherein the amount of venlafaxine, active metabolite or salt thereof released at 2 hours in 900 mL 0.1 N HCl (pH 1.5) with 40% EtOH using USP Apparatus II at 50 rpm is within 25%, within 15%, within 10%, or within 5% of the amount of venlafaxine, active metabolite or salt thereof released at 2 hours in 900 mL 0.1 N HCl (pH 1.5) using USP Apparatus II at 50 rpm.

In certain embodiments, the present invention is directed to a controlled release oral dosage form comprising: a therapeutically effective amount of venlafaxine, active metabolite of venlafaxine or a pharmaceutically acceptable salt thereof and a controlled release material; wherein the amount of venlafaxine, active metabolite or salt thereof released at 4 hours in 900 mL 0.1 N HCl (pH 1.5) with 40% EtOH using USP Apparatus II at 50 rpm is within 25%, within 15%, within 10%, or within 5% of the amount of venlafaxine, active metabolite or salt thereof released at 4 hours in 900 mL 0.1 N HCl (pH 1.5) using USP Apparatus II at 50 rpm.

In certain embodiments, the present invention is directed to a controlled release oral dosage form comprising: a therapeutically effective amount of venlafaxine, active metabolite of venlafaxine or a pharmaceutically acceptable salt thereof and a controlled release material; wherein the amount of venlafaxine, active metabolite or salt thereof released at 8 hours in 900 mL 0.1 N HCl (pH 1.5) with 40% EtOH using USP Apparatus II at 50 rpm is within 25%, within 15%, within 10%, or within 5% of the amount of venlafaxine, active metabolite or salt thereof released at 8 hours in 900 mL 0.1 N HCl (pH 1.5) using USP Apparatus II at 50 rpm.

In certain embodiments, the present invention is directed to a controlled release oral dosage form comprising: a therapeutically effective amount of venlafaxine, active metabolite of venlafaxine or a pharmaceutically acceptable salt thereof and a controlled release material; wherein the amount of venlafaxine, active metabolite or salt thereof released at 16 hours in 900 mL 0.1 N HCl (pH 1.5) with 40% EtOH using USP Apparatus II at 50 rpm is within 25%, within 15%, within 10%, or within 5% of the amount of venlafaxine, active metabolite or salt thereof released at 16 hours in 900 mL 0. I N HCl (pH 1.5) using USP Apparatus II at 50 rpm.

In certain embodiments, the present invention is directed to a controlled release oral dosage form comprising: a therapeutically effective amount of venlafaxine, active metabolite of venlafaxine or a pharmaceutically acceptable salt thereof and a controlled release material; wherein the amount of venlafaxine, active metabolite or salt thereof released at 24 hours in 900 mL 0.1 N HCl (pH 1.5) with 40% EtOH using USP Apparatus II at 50 rpm is within 25%, within 15%, within 10%, or within 5% of the amount of venlafaxine, active metabolite or salt thereof released at 24 hours in 900 mL 0. I N HCl (pH 1.5) using USP Apparatus II at 50 rpm.

In certain embodiments, the present invention is directed to a controlled release oral dosage form comprising: a therapeutically effective amount of venlafaxine, active metabolite or a pharmaceutically acceptable salt thereof and a controlled release material; wherein the ratio of the amount of venlafaxine, active metabolite or salt thereof released at I hour in 900 mL 0. I N HCl (pH 1.5) with 40% EtOH using USP Apparatus II at 50 rpm to the amount of venlafaxine, active metabolite or salt thereof released at I hour in 900 mL 0.1 N HCl (pH 1.5) using USP Apparatus II at 50 rpm is no more than 1.5:1; no more than about 1.2:1.

In certain embodiments, the present invention is directed to a controlled release oral dosage form comprising: a therapeutically effective amount of venlafaxine, active metabolite of venlafaxine or a pharmaceutically acceptable salt thereof and a controlled release material; wherein the ratio of the amount of venlafaxine, active metabolite or salt thereof released at 2 hours in 900 mL 0.1 N HCl (pH 1.5) with 40% EtOH using USP Apparatus 11 at 50 rpm to the amount of venlafaxine, active metabolite or salt thereof released at 2 hours in 900 mL 0.1 N HCl (pH 1.5) using USP Apparatus 11 at 50 rpm is no more than 1.5: 1; or no more than about 1.2:1.

In certain embodiments, the present invention is directed to a controlled release oral dosage form comprising a therapeutically effective amount of venlafaxine, active metabolite of venlafaxine or a pharmaceutically acceptable salt thereof and a controlled release material; wherein the ratio of the amount of venlafaxine, active metabolite or salt thereof released at 4 hours in 900 mL 0.1 N HCl (pH 1.5) with 40% EtOH using USP Apparatus 11 at 50 rpm to the amount of venlafaxine, active metabolite or salt thereof released at 4 hours in 900 mL 0.1 N HCl (pH 1.5) using USP Apparatus II at 50 rpm is no more than 1.5:1; no more than about 1.2:1.

In certain embodiments, the present invention is directed to a controlled release oral dosage form comprising a therapeutically effective amount of venlafaxine, active metabolite of venlafaxine or a pharmaceutically acceptable salt thereof and a controlled release material; wherein the ratio of the amount of venlafaxine, active metabolite or salt thereof released at 8 hours in 900 mL 0.1 N HCl (pH 1.5) with 40% EtOH using USP Apparatus 11 at 50 rpm to the amount of venlafaxine, active metabolite or salt thereof released at 8 hours in 900 mL 0.1 N HCl (pH 1.5) using USP Apparatus II at 50 rpm is no more than 1.5: 1; or no more than about 1.2:1.

In certain embodiments, the present invention is directed to a controlled release oral dosage form comprising a therapeutically effective amount of venlafaxine, active metabolite of venlafaxine or a pharmaceutically acceptable salt thereof and a controlled release material; wherein the ratio of the amount of venlafaxine, active metabolite or salt thereof released at 16 hours in 900 mL 0.1 N HCl (pH 1.5) with 40% EtOH using USP Apparatus II at 50 rpm to the amount of venlafaxine, active metabolite or salt thereof released at 16 hours in 900 mL 0.1 N HCl (pH 1.5) using USP Apparatus II at 50 rpm is no more than 1.5:1; or no more than about 1.2:1.

In certain embodiments, the present invention is directed to a controlled release oral dosage form comprising a therapeutically effective amount of venlafaxine, active metabolite of venlafaxine or a pharmaceutically acceptable salt thereof and a controlled release material; wherein the ratio of the amount of venlafaxine, active metabolite or salt thereof released at 24 hours in 900 mL 0.1 N HCl (pH 1.5) with 40% EtOH using USP Apparatus II at 50 rpm to the amount of venlafaxine, active metabolite or salt thereof released at 24 hours in 900 mL 0.1 N HCl (pH 1.5) using USP Apparatus II at 50 rpm is no more than 1.5:1; or no more than about 1.2:1.

In certain embodiments, the present invention is directed to a controlled release oral dosage form comprising a therapeutically effective amount of venlafaxine, an active metabolite of venlafaxine or a pharmaceutically acceptable salt thereof and a controlled release material; wherein the amount of venlafaxine, active metabolite or salt thereof released at 1 hour in 900 mL 0.1 N HCl (pH 1.5) with 40% EtOH using USP Apparatus II at 50 rpm is less than the amount of venlafaxine, active metabolite or salt thereof released at I hour in 900 mL 0.1 N HCl (pH 1.5) using USP Apparatus II at 50 rpm.

In certain embodiments, the present invention is directed to a controlled release oral dosage form comprising a therapeutically effective amount of venlafaxine, an active metabolite of venlafaxine or a pharmaceutically acceptable salt thereof and a controlled release material; wherein the amount of venlafaxine, active metabolite or salt thereof released at 2 hours in 900 mL 0.1 N HCl (pH 1.5) with 40% EtOH using USP Apparatus II at 50 rpm is less than the amount of venlafaxine, active metabolite or salt thereof released at 2 hours in 900 mL 0.1 N HCl (pH 1.5) using USP Apparatus II at 50 rpm.

In certain embodiments, the present invention is directed to a controlled release oral dosage form comprising a therapeutically effective amount of venlafaxine, an active metabolite of venlafaxine or a pharmaceutically acceptable salt thereof and a controlled release material; wherein the amount of venlafaxine, active metabolite or salt thereof released at 4 hours in 900 mL 0.1 N HCl (pH 1.5) with 40% EtOH using USP Apparatus II at 50 rpm is less than the amount of venlafaxine, active metabolite or salt thereof released at 4 hours in 900 mL 0.1 N HCl (pH 1.5) using USP Apparatus II at 50 rpm.

In certain embodiments, the present invention is directed to a controlled release oral dosage form comprising a therapeutically effective amount of venlafaxine, an active metabolite of venlafaxine or a pharmaceutically acceptable salt thereof and a controlled release material; wherein the amount of venlafaxine, active metabolite or salt thereof released at 8 hours in 900 mL 0.1 N HCl (pH 1.5) with 40% EtOH using USP Apparatus II at 50 rpm is less than the amount of venlafaxine, active metabolite or salt thereof released at 8 hours in 900 mL 0. I N HCl (pH 1.5) using USP Apparatus II at 50 rpm.

In certain embodiments, the present invention is directed to a controlled release oral dosage form comprising a therapeutically effective amount of venlafaxine, an active metabolite of venlafaxine or a pharmaceutically acceptable salt thereof and a controlled release material; wherein the amount of venlafaxine, active metabolite or salt thereof released at 16 hours in 900 mL 0.1 N HCl (pH 1.5) with 40% EtOH using USP Apparatus II at 50 rpm is less than the amount of venlafaxine, active metabolite or salt thereof released at 16 hours in 900 mL 0. I N HCl (pH 1.5) using USP Apparatus 11 at 50 rpm.

In certain embodiments, the present invention is directed to a controlled release oral dosage form comprising a therapeutically effective amount of venlafaxine, an active metabolite of venlafaxine or a pharmaceutically acceptable salt thereof and a controlled release material; wherein the amount of venlafaxine, active metabolite or salt thereof released at 24 hours in 900 mL 0.1 N HCl (pH 1.5) with 40% EtOH using USP Apparatus II at 50 rpm is less than the amount of venlafaxine, active metabolite or salt thereof released at 24 hours in 900 mL 0.1 N HCl (pH 1.5) using USP Apparatus II at 50 rpm.

In certain embodiments, the present invention is directed to a controlled release oral dosage form comprising a therapeutically effective amount of venlafaxine, an active metabolite of venlafaxine or a pharmaceutically acceptable salt thereof and a controlled release material; wherein the amount of venlafaxine, active metabolite or salt thereof released at 1 hour in 900 mL 0.1 N HCl (pH 4.5) with 40% EtOH using USP Apparatus II at 50 rpm is within 25%, within 15%, within 10%, or within 5% of the amount of venlafaxine, active metabolite or salt thereof released at 1 hour in 900 mL 0.1 N HCl (pH 4.5) using USP Apparatus II at 50 rpm.

In certain embodiments, the present invention is directed to a controlled release oral dosage form comprising: a therapeutically effective amount of venlafaxine, an active metabolite of venlafaxine or a pharmaceutically acceptable salt thereof and a controlled release material; wherein the amount of venlafaxine, active metabolite or salt thereof released at 2 hours in 900 mL 0.1 N HCl (pH 4.5) with 40% EtOH using USP Apparatus 11 at 50 rpm is within 25%, within 15%, within 10%, or within 5% of the amount of venlafaxine, active metabolite or salt thereof released at 2 hours in 900 mL 0.1 N HCl (pH 4.5) using USP Apparatus II at 50 rpm.

In certain embodiments, the present invention is directed to a controlled release oral dosage form comprising: a therapeutically effective amount of venlafaxine, an active metabolite of venlafaxine or a pharmaceutically acceptable salt thereof and a controlled release material; wherein the amount of venlafaxine, active metabolite or salt thereof released at 4 hours in 900 mL 0.1 N HCl (pH 4.5) with 40% EtOH using USP Apparatus II at 50 rpm is within 25%, within 15%, within 10%, or within 5% of the amount of venlafaxine, active metabolite or salt thereof released at 4 hours in 900 mL 0.1 N HCl (pH 4.5) using USP Apparatus II at 50 rpm.

In certain embodiments, the present invention is directed to a controlled release oral dosage form comprising: a therapeutically effective amount of venlafaxine, an active metabolite of venlafaxine or a pharmaceutically acceptable salt thereof and a controlled release material; wherein the amount of venlafaxine, active metabolite or salt thereof released at 8 hours in 900 mL 0.1 N HCl (pH 4.5) with 40% EtOH using USP Apparatus II at 50 rpm is within 25%, within 15%, within 10%, or within 5% of the amount of venlafaxine, active metabolite or salt thereof released at 8 hours in 900 mL 0.1 N HCl (pH 4.5) using USP Apparatus II at 50 rpm.

In certain embodiments, the present invention is directed to a controlled release oral dosage form comprising: a therapeutically effective amount of venlafaxine, an active metabolite of venlafaxine or a pharmaceutically acceptable salt thereof and a controlled release material; wherein the amount of venlafaxine, active metabolite or salt thereof released at 16 hours in 900 mL 0.1 N HCl (pH 4.5) with 40% EtOH using USP Apparatus 11 at 50 rpm is within 25%, within 15%, within 10%, or within 5% of the amount of venlafaxine, active metabolite or salt thereof released at 16 hours in 900 mL 0.1 N HCl (pH 4.5) using USP Apparatus 11 at 50 rpm.

In certain embodiments, the present invention is directed to a controlled release oral dosage form comprising: a therapeutically effective amount of venlafaxine, an active metabolite of venlafaxine or a pharmaceutically acceptable salt thereof and a controlled release material; wherein the amount of venlafaxine, active metabolite or salt thereof released at 24 hours in 900 mL 0.1 N HCl (pH 4.5) with 40% EtOH using USP Apparatus 11 at 50 rpm is within 25%, within 15%, within 10%, or within 5% of the amount of venlafaxine, active metabolite or salt thereof released at 24 hours in 900 mL 0.1 N HCl (pH 4.5) using USP Apparatus 11 at 50 rpm.

In certain embodiments, the present invention is directed to a controlled release oral dosage form comprising: a therapeutically effective amount of venlafaxine, an active metabolite of venlafaxine or a pharmaceutically acceptable salt thereof and a controlled release material; wherein the ratio of the amount of venlafaxine, active metabolite or salt thereof released at 1 hour in 900 mL 0.1 N HCl (pH 4.5) with 40% EtOH using USP Apparatus 11 at 50 rpm to the amount of venlafaxine, active metabolite or salt thereof released at I hour in 900 mL 0.1 N HCl (pH 4.5) using USP Apparatus 11 at 50 rpm is no more than 1.5:1; or no more than about 1.2:1.

In certain embodiments, the present invention is directed to a controlled release oral dosage form comprising: a therapeutically effective amount of venlafaxine, an active metabolite of venlafaxine or a pharmaceutically acceptable salt thereof and a controlled release material; wherein the ratio of the amount of venlafaxine, active metabolite or salt thereof released at 2 hours in 900 mL 0.1 N HCl (pH 4.5) with 40% EtOH using USP Apparatus 11 at 50 rpm to the amount of venlafaxine, active metabolite or salt thereof released at 2 hours in 900 mL 0.1 N HCl (pH 4.5) using USP Apparatus 11 at 50 rpm is no more than 1.5:1; or no more than about 1.2:1.

In certain embodiments, the present invention is directed to a controlled release oral dosage form comprising a therapeutically effective amount of venlafaxine, an active metabolite of venlafaxine or a pharmaceutically acceptable salt thereof and a controlled release material; wherein the ratio of the amount of venlafaxine, active metabolite or salt thereof released at 4 hours in 900 mL 0.1 N HCl (pH 4.5) with 40% EtOH using USP Apparatus II at 50 rpm to the amount of venlafaxine, active metabolite or salt thereof released at 4 hours in 900 mL 0.1 N HCl (pH 4.5) using USP Apparatus II at 50 rpm is no more than 1.5:1; or no more than about 1.2:1.

In certain embodiments, the present invention is directed to a controlled release oral dosage form comprising a therapeutically effective amount of venlafaxine, an active metabolite of venlafaxine or a pharmaceutically acceptable salt thereof and a controlled release material; wherein the ratio of the amount of venlafaxine, active metabolite or salt thereof released at 8 hours in 900 mL 0.1 N HCl (pH 4.5) with 40% EtOH using USP Apparatus II at 50 rpm to the amount of venlafaxine, active metabolite or salt thereof released at 8 hours in 900 mL 0.1 N HCl (pH 4.5) using USP Apparatus II at 50 rpm is no more than 1.5: 1; or no more than about 1.2:1.

In certain embodiments, the present invention is directed to a controlled release oral dosage form comprising a therapeutically effective amount of venlafaxine, an active metabolite of venlafaxine or a pharmaceutically acceptable salt thereof and a controlled release material; wherein the ratio of the amount of venlafaxine, active metabolite or salt thereof released at 16 hours in 900 mL 0.1 N HCl (pH 4.5) with 40% EtOH using USP Apparatus II at 50 rpm to the amount of venlafaxine, active metabolite or salt thereof released at 16 hours in 900 mL 0.1 N HCl (pH 4.5) using USP Apparatus II at 50 rpm is no more than 1.5:1; or no more than about 1.2:1.

In certain embodiments, the present invention is directed to a controlled release oral dosage form comprising a therapeutically effective amount of venlafaxine, an active metabolite of venlafaxine or a pharmaceutically acceptable salt thereof and a controlled release material; wherein the ratio of the amount of venlafaxine, active metabolite or salt thereof released at 24 hours in 900 mL 0.1 N HCl (pH 4.5) with 40% EtOH using USP Apparatus II at 50 rpm to the amount of venlafaxine, active metabolite or salt thereof released at 24 hours in 900 mL 0.1 N HCl (pH 4.5) using USP Apparatus II at 50 rpm is no more than 1.5:1;ornomorethan about 1.2:1.

In certain embodiments, the present invention is directed to a controlled release oral dosage form comprising a therapeutically effective amount of venlafaxine, an active metabolite of venlafaxine or a pharmaceutically acceptable salt thereof and a controlled release material; wherein the amount of venlafaxine, active metabolite or salt thereof released at I hour in 900 mL 0.1 N HCl (pH 4.5) with 40% EtOH using USP Apparatus II at 50 rpm is less than the amount of venlafaxine, active metabolite or salt thereof released at I hour in 900 mL 0.1 N HCl (pH 4.5) using USP Apparatus II at 50 rpm.

In certain embodiments, the present invention is directed to a controlled release oral dosage form comprising a therapeutically effective amount of venlafaxine, an active metabolite of venlafaxine or a pharmaceutically acceptable salt thereof and a controlled release material; wherein the amount of venlafaxine, active metabolite or salt thereof released at 2 hours in 900 mL 0.1 N HCl (pH 4.5) with 40% EtOH using USP Apparatus II at 50 rpm is less than the amount of venlafaxine, active metabolite or salt thereof released at 2 hours in 900 mL 0.1 N HCl (pH 4.5) using USP Apparatus II at 50 rpm.

In certain embodiments, the present invention is directed to a controlled release oral dosage form comprising a therapeutically effective amount of venlafaxine, an active metabolite of venlafaxine or a pharmaceutically acceptable salt thereof and a controlled release material; wherein the amount of venlafaxine, active metabolite or salt thereof released at 4 hours in 900 mL 0.1 N HCl (pH 4.5) with 40% EtOH using USP Apparatus II at 50 rpm is less than the amount of venlafaxine, active metabolite or salt thereof released at 4 hours in 900 mL 0.1 N HCl (pH 4.5) using USP Apparatus II at 50 rpm.

In certain embodiments, the present invention is directed to a controlled release oral dosage form comprising a therapeutically effective amount of venlafaxine, an active metabolite of venlafaxine or a pharmaceutically acceptable salt thereof and a controlled release material; wherein the amount of venlafaxine, active metabolite or salt thereof released at 8 hours in 900 mL 0.1 N HCl (pH 4.5) with 40% EtOH using USP Apparatus II at 50 rpm is less than the amount of venlafaxine, active metabolite or salt thereof released at 8 hours in 900 mL 0.1 N HCl (pH 4.5) using USP Apparatus II at 50 rpm.

In certain embodiments, the present invention is directed to a controlled release oral dosage form comprising a therapeutically effective amount of venlafaxine, an active metabolite of venlafaxine or a pharmaceutically acceptable salt thereof and a controlled release material; wherein the amount of venlafaxine, active metabolite or salt thereof released at 16 hours in 900 mL 0.1 N HCl (pH 4.5) with 40% EtOH using USP Apparatus II at 50 rpm is less than the amount of venlafaxine, active metabolite or salt thereof released at 16 hours in 900 mL 0.1 N HCl (pH 4.5) using USP Apparatus II at 50 rpm.

In certain embodiments, the present invention is directed to a controlled release oral dosage form comprising a therapeutically effective amount of venlafaxine, an active metabolite of venlafaxine or a pharmaceutically acceptable salt thereof and a controlled release material; wherein the amount of venlafaxine, active metabolite or salt thereof released at 24 hours in 900 mL 0.1 N HCl (pH 4.5) with 40% EtOH using USP Apparatus II at 50 rpm is less than the amount of venlafaxine, active metabolite or salt thereof released at 24 hours in 900 mL 0.1 N HCl (pH 4.5) using USP Apparatus II at 50 rpm.

In certain embodiments, the dosage forms of the present invention are dose proportional or substantially dose proportional.

In certain embodiments, the dosage forms of the present invention are pseudo dose proportional.

In certain embodiments, the dosage form of the present invention does not comprise carboxymethylcellulose.

In certain embodiments of the present invention, the gelling agent is included in an amount from about 40% to about 70%, and more preferably from about 50% to about 65%, by weight of the final product. The drug to gum ratio may be, e.g., from about 1:0.5 to about 1:6. More preferably, the drug to gum ratio is from about 1:2.1 to about 1:8.4.

In certain embodiments, the controlled release dosage form of the present invention comprises an effective amount of an ionizable gel strength enhancing agent to obtain a desirable increased gel strength due to cross-linking with the gelling agent.

In certain embodiments, the controlled release dosage form of the present invention further comprises a hydrophobic material in an amount effective to slow the hydration of the gelling agent when the formulation is exposed to an environmental fluid.

In certain preferred embodiments, the present invention is further directed to a controlled release oral tablet comprising venlafaxine, an active metabolite of venlafaxine or a pharmaceutically acceptable salt thereof (e.g., venlafaxine hydrochloride) in an amount, e.g., from about 25 mg to about 500 mg or from about 75 mg to about 375 mg, dispersed in a matrix comprising (i) a gelling agent, the gelling agent in an amount of from about 20% to about 70% by weight of the dosage form, (ii) an inert pharmaceutical diluent in an amount of from about 5% to about 40% by weight of the dosage form, and (iii) an ionizable gel strength enhancing agent in an amount of from about 4% to about 10% by weight of the dosage form; a hydrophobic coating coated over the matrix in an amount of from about 1% to about 20% by weight of the dosage form; wherein the formulation provides for the controlled release of the agent and is suitable for once-a-day administration.

In certain embodiments, the dosage forms of the present invention comprise venlafaxine, an active metabolite of venlafaxine or a pharmaceutically acceptable salt thereof (e.g., venlafaxine hydrochloride) in an amount, e.g., from about 25 mg to about 500 mg or from about 75 mg to about 375 mg, dispersed in a controlled release excipient comprising (i) locust bean gum in an amount of 10% to about 40% by weight, (ii) xanthan gum in an amount from about 10% to about 30% by weight, (iii) mannitol in an amount of from about 5% to about 40% by weight, and (iv) calcium sulfate dihydrate in an amount of about 4% to about 10% by weight.

In certain embodiments, the dosage form can comprise a hydrophobic material coated over the matrix in an amount, e.g., from amount about 1% to about 25% in order to provide a further controlled release or to provide a delayed release with e.g., an enteric coating.

In certain embodiments, the controlled release dosage form of the present invention comprises from about 7% to about 35% venlafaxine, active metabolite or salt thereof by weight of the dosage form or from about 15% to about 30% of the dosage form.

In certain embodiments, the controlled release dosage form of the present invention comprises from about 35% to about 85% gelling agent by weight of the dosage form or from about 50% to about 70% by weight of the dosage form.

In certain embodiments, the controlled release dosage form of the present invention comprises from about 1% to about 25% hydrophobic material by weight of the dosage form, or from about 5% to about 15% hydrophobic material by weight of the dosage form.

In certain embodiments the invention is directed to a controlled release oral dosage form comprising a matrix comprising therapeutically effective amount of venlafaxine, active metabolite of venlafaxine or a pharmaceutically acceptable salt thereof dispersed in a gelling agent; the gelling agent comprising a heteropolysaccharide gum; and the matrix providing a controlled release of the venlafaxine, active metabolite of venlafaxine or a pharmaceutically acceptable salt thereof, to provide therapeutic plasma levels of venlafaxine for about 12 to about 30 hours after oral administration to human patients.

In certain embodiments the invention is directed to a controlled release oral dosage form comprising: a matrix comprising therapeutically effective amount of an active metabolite of venlafaxine or a pharmaceutically acceptable salt thereof dispersed in a gelling agent; the gelling agent comprising a heteropolysaccharide gum; and the matrix providing a controlled release of the active agent to provide 24 hour therapeutic plasma levels of the active metabolite of venlafaxine after oral administration to human patients.

In certain embodiments the invention is directed to a controlled release oral dosage form comprising: venlafaxine or a pharmaceutically acceptable salt thereof and at least one controlled release excipient; the dosage form provides a mean ratio of Cmax of venlafaxine to I mg of venlafaxine base of greater than 1:1; greater than 1:1.1; greater than 1:1.2; or greater than 1:1.3. In certain embodiments, the dosage form also provides a mean ratio of Cmax of venlafaxine to 1 mg of venlafaxine base of less than 1:2 or less than less than 1:1.5.

In certain embodiments the invention is directed to a controlled release oral dosage form comprising: an active metabolite of venlafaxine or a pharmaceutically acceptable salt thereof and at least one controlled release excipient; the dosage form provides a mean ratio of Cmax of active metabolite of venlafaxine to I mg of active metabolite of venlafaxine base of greater than 1:1; greater than 1:1.1; greater than 1:1.2; or greater than 1:1.3. In certain embodiments, the dosage form also provides a mean ratio of Cmax of active metabolite of venlafaxine to I mg of active metabolite of venlafaxine base of less than 1:2 or less than less than 1:1.5.

The formulations of the present invention which are resistant to alcohol induced dose dumping are not limited to dosage forms comprising a gelling agent. Any suitable controlled release technology which can provide the required resistance can be used. Such controlled release technology is described, e.g., in U.S. Patent Publication Nos. 2003/0118641; 2005/0163856; and 2004/0052731, the disclosures of which are hereby incorporated by reference.

In certain embodiments, the active metabolite of venlafaxine utilized in the formulations disclosed herein is preferably O-desmethyl-venlafaxine or a pharmaceutically acceptable salt thereof. In certain embodiments, the salt is the formate or succinate salt.

In certain embodiments the invention is directed to a controlled release oral dosage form comprising venlafaxine; an active metabolite of venlafaxine; or a pharmaceutically acceptable salt thereof; wherein the dosage form is scored in order to divide the dosage form into equal or substantially equal divided doses.

In certain embodiments the invention is directed to a controlled release oral dosage form comprising a matrix comprising (i) venlafaxine, an active metabolite of venlafaxine or a pharmaceutically acceptable salt thereof and (ii) at least one controlled release excipient; the dosage form being scored in order to divide the dosage form into at least two substantially equal divided doses.

In certain embodiments the invention is directed to a method of titrating a patient in need of venlafaxine therapy comprising: a) dividing a scored dosage form as disclosed herein into divided doses; b) administering a divided dose for at least one dosing interval to the patient; and c) increasing the dosage in a subsequent administration.

In certain embodiments the invention is directed to a method of titrating a patient in need of venlafaxine therapy comprising: a) dividing a scored dosage form as disclosed herein into divided doses; and b) administering to a patient currently on venlafaxine therapy a divided dose for at least one dosing interval in order to decrease the dosage to the patient.

In certain embodiments, the titration therapy disclosed herein results in decreased side effects associated with venlafaxine therapy, e.g., nausea and/or vomiting as compared to immediate release therapy; as compared to initially administering the intended final dosage of venlafaxine; or as compared to abrupt cessation of the venlafaxine therapy.

By “controlled” it is meant for purposes of the present invention that the therapeutically active medicament is released from the formulation at a controlled rate such that therapeutically beneficial blood levels (but below toxic levels) of the medicament are maintained over an extended period of time, e.g., providing a 24 hour therapeutic effect.

The term “environmental fluid” is meant for purposes of the present invention to encompass, e.g., an aqueous solution, such as that used for in-vitro dissolution testing, or gastrointestinal fluid.

The term “Cmax” is meant for purposes of the present invention to mean the maximum plasma concentration of a medicament achieved after single dose administration of a dosage form in accordance with the present invention. The term “Cₘₐₓ at steady state” is meant for purposes of the present invention to mean the maximum plasma concentration of a medicament achieved after state administration of a dosage form in accordance with the present invention.

The term “human subject” for purposes of the present invention is a healthy human volunteer naive to venlafaxine, an active metabolite of venlafaxine or a pharmaceutically acceptable salt thereof.

The term “human patient” for purposes of the present invention is a human in need of treatment with venlafaxine, an active metabolite of venlafaxine or a pharmaceutically acceptable salt thereof, therapy.

The term “Tₘₐₓ” is meant for purposes of the present invention to mean the elapsed time from administration of a dosage form to the time the Cmax of the medicament is achieved.

The term “mean” for purposes of the present invention, when used to define a pharmacokinetic value (e.g., Tₘₐₓ) represents the arithmetic mean value measured across a patient population.

The term “dose proportional” for purposes of the present invention means that all active and inactive ingredients are in exactly the same proportion between different strengths (e.g., a tablet of 75-mg strength has all the inactive ingredients, exactly half that of a tablet of 150-mg strength, and twice that of a tablet of 37.5-mg strength).

The term “pseudo-dose proportional” means that either 1) the portion of the reduced active ingredient amount in the lower strength dosage form is replaced by an inert diluent such that the total tablet weight is same and the ratios of the inactive ingredients to total tablet weight except the inert diluent is the same or 2) the portion of the reduced active ingredient amount in the lower strength dosage form is not replaced by an inert diluent such that the total tablet weight is reduced equal to the lesser active ingredient and the ratios of the inactive ingredients to total tablet weight are the not the same.

The term “venlafaxine therapy” means therapy utilizing venlafaxine, an active metabolite of venlafaxine or a pharmaceutically acceptable salt thereof.

The term “to provide 24 hour therapeutic plasma levels after oral administration to human patients” means that the dosage forms provides suitable blood levels which allow for once-a-day administration. It is recognized to one skilled in the art that a single or initial dose of the dosage forms disclosed herein may not necessarily provide therapeutic blood levels for a full 24 hours as there may be a lag time to reach therapeutic levels as after administration, the agent must dissolve into gastrointestinal fluid and be absorbed through mucous membranes into the circulatory system.

The term “active metabolite” means active metabolite of venlafaxine.

The term “salt” means pharmaceutically acceptable salt thereof.

### BRIEF DESCRIPTION OF THE DRAWINGS

FIG. 1 shows the dissolution rates of the formulations of Examples 19-21.

FIG. 2 shows the dissolution rates for Example 22A.

FIG. 3 shows the dissolution rates for Effexor® XR 37.5 mg capsules.

FIG. 4 shows the dissolution rates for Example 22B.

FIG. 5 shows the dissolution rates for Effexor® XR 75 mg capsules.

FIG. 6 shows the dissolution rates for Example 22C.

FIG. 7 shows the dissolution rates for Effexor® XR 150 mg capsules.

FIG. 8 compares the results of Examples 23A-F with the following parameters: Apparatus—USP Type II; Volume—900 ml; Rotational Speed—50 rpm; Media—40% Ethanol in 0.1 N HCl.

FIG. 9 compares the results of Examples 23A-F with the following parameters: Apparatus—USP Type II; Volume—900 ml; Rotational Speed—50 rpm; Media—40% Ethanol in pH 4.5.

### DETAILED DESCRIPTION

Venlafaxine has been shown to be a potent inhibitor of monoamine neurotransmitter uptake, a mechanism associated with clinical antidepressant activity. Due to its novel structure, venlafaxine has a mechanism of action unrelated to other available antidepressants, such as the tricyclic antidepressants desipramine, nortriptyline, protriptyline, imipramine, amitryptyline, trimipramine and doxepin.

It is believed that venlafaxine's mechanism of action is related to potent inhibition of the uptake of the monoamine neurotransmitters serotonin and norepinephrine. To a lesser degree, venlafaxine also inhibits dopamine reuptake, but it has no inhibitory activity on monoamine oxidase. O-desmethyl-venlafaxine, venlafaxine's major metabolite in humans, exhibits a similar pharmacologic profile. Venlafaxine's ability to inhibit norepinephrine and serotonin (5-HT) uptake has been predicted to have an efficacy which rivals or surpasses that of tricyclic antidepressants (Stuart A. Montgomery, M. D., J. Clin. Psychiatry, 54:3, March 1993).

Venlafaxine has been marketed as a once-a-day controlled release dosage form (i.e. Effexor XR) wherein the total daily dose is contained in one dosage form, with the intent that the agent is slowly released over the dosing interval. This dosage form, however, is susceptible to alcohol induced dose dumping, whereby the integrity of the controlled release mechanism is compromised in the presence of alcohol. This can result in an immediate release of the contents of the dosage form which was intended to release slowly over the entire dosing interval. This can result in “spiked” plasma levels of venlafaxine and may result in increased side effects to the patient. In certain embodiments, the dosage forms of the present invention address this need in the art for a controlled release formulation of venlafaxine, active metabolite of venlafaxine, or salt thereof, which is resistant to alcohol induced dose dumping.

The preferred venlafaxine salt of the present invention is venlafaxine hydrochloride in an amount e.g., equivalent to about 37.5 mg base, about 75 mg base or about 150 mg base.

The preferred active metabolite of the present invention is O-desmethyl-venlafaxine in salt form such as 0-desmethyl-venlafaxine formate or O-desmethyl-venlafaxine succinate in an amount that would be e.g., therapeutically equivalent to about 37.5 mg base, about 75 mg base or about 150 mg base.

In certain embodiments, the magnitude of a prophylactic or therapeutic dose of O-desmethyl-venlafaxine or salt thereof, in the acute or chronic management of a disease will vary with the severity of the condition to be treated and the route of administration. The dose will also vary according to age, body weight, response, and the past medical history of the individual patient. In general the daily dose will lie within the range of from about 10 mg to about 1000 mg per day. Preferably, a daily dose range should be from about 50 mg to about 500 mg per day, more preferably, between about 75 mg and about 350 mg per day.

In certain embodiments where an O-desmethyl-venlafaxine salt is utilized, the daily dose will lie within the range of the salt form that is equivalent to from about 10 mg to about 1000 mg per day; preferably from about 50 mg to about 500 mg per day; and more preferably, between about 75 mg and about 350 mg per day.

In certain embodiments, the controlled release dosage form comprises 0-desmethyl-venlafaxine formate in an amount equivalent to from about 10 mg to about 1000 mg O-desmethyl-venlafaxine base.

In certain preferred embodiments, the controlled release dosage form comprises O-desmethyl-venlafaxine formate in an amount equivalent to from about 50 mg to about 500 mg O-desmethyl-venlafaxine base.

In certain more preferred embodiments, the controlled release dosage form comprises O-desmethyl-venlafaxine formate in an amount equivalent to from about 75 mg to about 375 mg O-desmethyl-venlafaxine base.

In certain embodiments, the controlled release dosage form comprises O-desmethyl-venlafaxine succinate in an amount equivalent to from about 10 mg to about 1000 mg O-desmethyl-venlafaxine base.

In certain preferred embodiments, the controlled release dosage form comprises O-desmethyl-venlafaxine succinate in an amount equivalent to from about 50 mg to about 500 mg O-desmethyl-venlafaxine base.

In certain more preferred embodiments, the controlled release dosage form comprises O-desmethyl-venlafaxine succinate in an amount equivalent to from about 75 mg to about 350 mg O-desmethyl-venlafaxine base.

O-desmethyl-venlafaxine succinate is prepared as described in U.S. Pat. No. 6,673,838, which is incorporated by reference herein. The formate salt of O-desmethyl-venlafaxine, described in published U.S. Patent Application Publication No. U.S. 2003/0236309, which is incorporated by reference herein, can be prepared using similar techniques by substitution of the appropriate salt.

The polymorphs of O-desmethyl-venlafaxine succinate are described in U.S. patent application Publication No. U.S. 20050096479. Crystalline forms of O-desmethyl-venlafaxine formate are described in U.S. patent application Publication No. U.S. 20030236309. All of these publications are incorporated by reference.

The venlafaxine or active metabolite of venlafaxine utilized in the invention can include the free base and any pharmaceutically acceptable salt forms of the agent, the racemate and its individual enantiomers, and analogs, both as racemates and as their individual enantiomers. The use of venlafaxine or active metabolite of venlafaxine is also understood to include all crystalline and amorphous forms, and any polymorphs or hydrates.

The pharmaceutically acceptable salts of venlafaxine or active metabolites of venlafaxine include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like.

The polymorphs of venlafaxine hydrochloride include, but are not limited to Form C, which is a crystalline form of venlafaxine hydrochloride having a melting point between 215 and 217° C.; Form A; Form B; and Form D, which is a crystalline hydrate of venlafaxine hydrochloride. Other polymorphs of venlafaxine hydrochloride include orthorhombic crystal structures and monoclinic crystal structures. In addition, there are two solvate forms, which are described in US 2002/014321 A1 and WO 03/048082. Other polymorphs are described in US 2003/0109585A1 (an anhydrous form) and US 2003/0114536 A1 (a hydrous form). All of these publications are incorporated by reference.

In certain embodiments of the present invention, the gelling agent comprises a polysaccharide, such as a heteropolysaccharide or a homopolysaccharide.

In certain embodiments of the present invention, the gelling agent comprises a microbial polysaccharide such as xanthan gum and the like.

In a preferred embodiment, the gelling agent comprises a heteroplysaccharide and further comprises a homopolysaccharide gum capable of cross-linking the heteropolysaccharide gum when exposed to an environmental fluid. The preferred heteropolysaccharide gum is xanthan gum and the preferred homopolysaccharide gum is a galactomannan such as locust bean gum.

A non-limiting list of suitable hydrophobic materials which may be included in the dosage form of the present invention includes, gums, cellulose ethers, acrylic resins, protein derived materials, waxes, shellac, and oils such as hydrogenated castor oil and hydrogenated vegetable oil. Certain hydrophobic polymers include alkylcelluloses such as ethylcellulose, acrylic and methacrylic acid polymers and copolymers. Examples of acrylic and methacrylic acid polymers and copolymers include methyl methacrylate, methyl methacrylate copolymers, ethoxyethyl methacrylates, ethyl acrylate, trimethyl ammonioethyl methacrylate, cyanoethyl methacrylate, aminoalkyl methacrylate copolymer, poly(acrylic acid), poly(methacrylic acid), methacrylic acid alkylamine copolymer, poly(methyl methacrylate), poly(methacrylic acid)(anhydride), polymethacrylate, polyacrylamide, poly(methacrylic acid anhydride), and glycidyl methacrylate copolymers. Preferred waxes include for example natural and synthetic waxes, fatty acids, fatty alcohols, and mixtures of the same (e.g., beeswax, camauba wax, stearic acid and stearyl alcohol.

In certain preferred embodiments, the gelling agent of the present invention comprises a heteropolysaccharide such as xanthan gum, a homopolysaccharide such as locust bean gum, or a mixture of one or more hetero- and one or more homopolysaccharide(s). Heterodisperse excipients, previously disclosed in our U.S. Pat. Nos. 4,994,276, 5,128,143, and 5,135,757, may be utilized in the sustained release excipient of the present invention. For example, in certain embodiments of the present invention, the controlled release excipient comprises a gelling agent of both hetero- and homo- polysaccharides which exhibit synergism, e.g., the combination of two or more polysaccharide gums producing a higher viscosity and faster hydration than that which would be expected by either of the gums alone, the resultant gel being faster-forming and more rigid.

The term “heteropolysaccharide” as used in the present invention is defined as a water-soluble polysaccharide containing two or more kinds of sugar units, the heteropolysaccharide having a branched or helical configuration, and having excellent water-wicking properties and immense thickening properties.

An especially preferred heteropolysaccharide is xanthan gum, which is a high molecular weight (>1 06) heteropolysaccharide. Other preferred heteropolysaccharides include derivatives of xanthan gum, such as deacylated xanthan gum, the carboxymethyl ether, and the propylene glycol ester.

The homopolysaccharide materials used in the present invention that are capable of cross-linking with the heteropolysaccharide include the galactomannans, i.e., polysaccharides that are composed solely of mannose and galactose. A possible mechanism for the interaction between the galactomannan and the heteropolysaccharide involves the interaction between the helical regions of the heteropolysaccharide and the unsubstituted mannose regions of the galactomannan. Galactomannans that have higher proportions of unsubstituted mannose regions have been found to achieve more interaction with the heteropolysaccharide. Hence, locust bean gum, which has a higher ratio of mannose to galactose, is especially preferred.

The combination of xanthan gum with locust bean gum is an especially preferred gum combination for use in the sustained release excipient of the present invention.

In certain preferred embodiments of the present invention, the controlled release properties of the final product are optimized when the ratio of heteropolysaccharide gum to homopolysaccharide gum is from about 1:3 to about 3:1. However, the final product may comprise from about 1% to about 99% by weight heteropolysaccharide gum and from about 99% to about 1% by weight homopolysaccharide gum.

The combination of any homopolysaccharide gums know to produce a synergistic effect when exposed to aqueous solutions may be used in accordance with the present invention. It is also possible that the type of synergism which is present with regard to the gum combination of the present invention could also occur between two homogeneous or two heteropolysaccharides. Other acceptable gelling agents which may be used in the present invention include those gelling agents well-known in the art. Examples include vegetable gums such as alginates, carrageenan, pectin, guar gum, modified starch, hydroxypropylmethylcellulose, methylcellulose, and other cellulosic materials such as and hydroxypropyl cellulose. This list is not meant to be exclusive.

The dosage form of the present invention can further comprise an inert diluent. The inert diluent of the sustained release excipient preferably comprises a pharmaceutically acceptable saccharide, including a monosaccharide, a disaccharide, or a polyhydric alcohol, and/or mixtures of any of the foregoing. Examples of suitable inert pharmaceutical diluents include sucrose, dextrose, lactose, mannitol, microcrystalline cellulose, fructose, xylitol, sorbitol, starches, mixtures thereof and the like. However, it is preferred that a soluble pharmaceutical filler such as lactose, dextrose, mannitol, sucrose, or mixtures thereof be used. In certain especially preferred embodiments the diluent or filler is mannitol.

In certain embodiments, it is possible to dry mix the ingredients of the dosage form without utilizing a wet granulation step. This procedure may be utilized, for example, where a wet granulation is to be accomplished when the active ingredient is directly added to the ingredients of the sustained release excipient. On the other hand, this procedure may also be used where no wet granulation step whatsoever is contemplated. If the mixture is to be manufactured without a wet granulation step, and the final mixture is to be tableted, it is preferred that all or part of the inert diluent comprise a pre-manufactured direct compression diluent. Such direct compression diluents are widely used in the pharmaceutical arts, and may be obtained from a wide variety of commercial sources. Examples of such pre-manufactured direct compression excipients include Emcocel® (microcrystalline cellulose, N.F.), Emdex® (dextrates, N.F.), and Tab-Fine® (a number of direct-compression sugars including sucrose, fructose and dextrose), all of which are commercially available from JRS Pharma LP, Patterson, N.Y.). Other direct compression diluents include Anhydrous lactose (Lactose N.F., anhydrous direct tableting) from Sheffield Chemical, Union, N.J. 07083; Elcems® G-250 (powdered cellulose), N.F.) from Degussa, D-600 Frankfurt (Main) Germany; Fast-Flo Lactose® (Lactose, N.F., spray dried) from Foremost Whey Products, Banaboo, Wis. 53913; Maltrin® (Agglomerated maltodextrin) from Grain Processing Corp., Muscatine, Iowa 52761; Neosorb 60® (Sorbitol, N.F., direct-compression from Roquet Corp., 645 5th Ave., New York, N.Y. 10022; Nu-Tab® (Compressible sugar, N.F.) from Ingredient Technology, Inc., Pennsauken, N.J. 08110; Polyplasdone XL® (Crospovidone, N.F., cross-linked polyvinylpyrrolidone) from ISP Corp., Wayne, N.J. 07470; Primojel® (Sodium starch glycolate, N.F., carboxymethyl starch) from Generichem Corp., Little Falls, N.J. 07424; Solka Floc® (Cellulose floc); Spray-dried lactose® (Lactose N.F., spray dried) from Foremost Whey Products, Baraboo, Wis. 53913 and DMV Corp., Vehgel, Holland; and Sta-Rx 1500® (Starch 1500) (Pregelatinized starch, N.F., compressible) from Colorcon, Inc., West Point, Pa. 19486.

In further embodiments of the present invention, the directly compressible inert diluent which is used in conjunction with the sustained release excipient of the present invention is an augmented microcrystalline cellulose as disclosed in U.S. patent application Ser. No. 08/370,576, filed Jan. 9, 1995, and entitled “PHARMACEUTICAL EXClPIENT HAVING IMPROVED COMPRESSIBILITY”, by J. Staniforth, B. Sherwood and E. Hunter, hereby incorporated by reference in its entirety. The augmented microcrystalline cellulose described therein is commercially available under the tradename “Prosolv” from JRS Pharma LP.

The controlled release excipients prepared in accordance with the present invention may be prepared according to any agglomeration technique to yield an acceptable sustained release excipient product. In wet granulation techniques, the desired amounts of the heteropolysaccharide gum, the homopolysaccharide gum, and the inert diluent are mixed together and thereafter a moistening agent such as water, propylene glycol, glycerol, alcohol or the like is added to prepare a moistened mass. Next, the moistened mass is dried. The dried mass is then milled with conventional equipment into granules. Thereafter, the excipient product is ready to use. The granulate form has certain advantages including the fact that it can be optimized for flow and compressibility; it can be tableted, formulated in a capsule, extruded and spheronized with an active medicament to form pellets, etc.

In certain embodiments of the invention where the controlled release excipient comprises a heteropolysaccharide, a homopolysaccharide, or both, a release-modifying agent may also be incorporated in the formulations (e.g., in the sustained release excipient) of the present invention. Such release-modifying agents and pre-manufactured excipients disclosed in our U.S. Pat. Nos. 5,455,046; 5,512,297; 5,554,387; 5,667,801; 5,846,563; 5,773,025; 6,048,548; 5,662,933; 5,958,456; 5,472,711; 5,670,168; and 6,039,980 may be utilized in the present invention.

Thus, for example, the release-modifying agent may comprise an ionizable gel-strength enhancing agent. The ionizable gel strength-enhancing agent that is optionally used in conjunction with the present invention may be monovalent or multivalent metal cations. The preferred salts are the inorganic salts, including various alkali metal and/or alkaline earth metal sulfates, chlorides, borates, bromides, citrates, acetates, lactates, etc. Specific examples of suitable ionizable gel strength enhancing agent include calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, sodium fluoride, and mixtures thereof. Multivalent metal cations may also be utilized. However, the preferred ionizable gel strength-enhancing agents are bivalent. Particularly preferred salts are calcium sulfate and sodium chloride. In a particular preferred embodiment, the ionizable gel strength-enhancing agent is calcium sulfate dihydrate. The ionizable gel strength enhancing agents of the present invention are added in an amount effective to obtain a desirable increased gel strength due to the cross-linking of the gelling agent (e.g., the heteropolysaccharide and homopolysaccharide gums). In certain embodiments, the ionizable gel strength-enhancing agent is included in the controlled release excipient of the present invention in an amount from about I to about 20% by weight of the controlled release excipient, and in an amount 0.5% to about 16% by weight of the final dosage form.

In certain embodiments, the dosage form of the present invention can comprise a surfactant. Surfactants that may be used in the present invention generally include pharmaceutically acceptable anionic surfactants, cationic surfactants, amphoteric (amphipathic/ amphophilic) surfactants, and non-ionic surfactants. Suitable pharmaceutically acceptable anionic surfactants include, for example, monovalent alkyl carboxylates, acyl lactylates, alkyl ether carboxylates, N-acyl sarcosinates, polyvalent alkyl carbonates, N-acyl glutamates, fatty acid-polypeptide condensates, sulfuric acid esters, alkyl sulfates (including sodium lauryl sulfate (SLS)), ethoxylated alkyl sulfates, ester linked sulfonates (including docusate sodium or dioctyl sodium succinate (DSS)), alpha olefin sulfonates, and phosphated ethoxylated alcohols.

Suitable pharmaceutically acceptable cationic surfactants include, for example, monoalkyl quaternary ammonium salts, dialkyl quaternary ammonium compounds, amidoamines, and aminimides.

Suitable pharmaceutically acceptable amphoteric (amphipathic/amphophilic) surfactants, include, for example, N-substituted alkyl amides, N-alkyl betaines, sulfobetaines, and N-alkyl β-aminoproprionates.

Other suitable surfactants for use in conjunction with the present invention include polyethyleneglycols as esters or ethers. Examples include polyethoxylated castor oil, polyethoxylated hydrogenated castor oil, or polyethoxylated fatty acid from castor oil or polyethoxylated fatty acid from hydrogenated castor oil. Commercially available surfactants that can be used are known under trade names Cremophor, Myrj, Polyoxyl 40 stearate, Emerest 2675, Lipal 395 and PEG 3350.

Other release-modifying pharmaceutically acceptable agents that may be added in appropriate quantities for their particular ability to modify dissolution rates include, for example: stearic acid, metallic stearates, stearyl alcohol, hydrogenated cotton seed oil, sodium chloride and certain disintegrants.

The quantity of such release-modifying agent employed depends on the release characteristics required and the nature of the agent. For the sustained release formulation according to the invention, the level of release-modifying agents used may be from about 0.1 to about 25%, preferably from about 0.5 to about 20% by weight of the total composition.

In certain embodiments, the dosage form of the present invention can comprise suitable quantities of additional excipients, e.g., lubricants, binders, granulating aids, diluents, colorants, flavorants and glidants which are conventional in the pharmaceutical art.

Specific examples of pharmaceutically acceptable diluents and excipients that may be used in formulating the cores are described in the Handbook of Pharmaceutical Excipients, American Pharmaceutical Association (1986), incorporated by reference herein.

Examples of suitable binders for use in the present invention include for example and without limitation, povidone, polyvinylpyrrolidone, xanthan gum, cellulose gums such as carboxymethylcellulose, methyl cellulose, hydroxypropylmethylcellulose, hydroxycellulose, gelatin, starch, and pregelatinized starch.

In certain preferred embodiments, the binder is hypromellose.

In certain embodiments of the present invention a hydrophobic material is added to the formulation. This may be accomplished by granulating the sustained release excipient with a solution or dispersion of hydrophobic material prior to the incorporation of the medicament. The hydrophobic material may be selected from ethylcellulose, acrylic and/or methacrylic acid polymers or copolymers, hydrogenated vegetable oils, zein, as well as other pharmaceutically acceptable hydrophobic materials known to those skilled in the art. Other hydrophobic cellulosic materials such as other alkyl celluloses may also be used. The amount of hydrophobic material incorporated into the sustained release excipient is that which is effective to slow the hydration of the gums without disrupting the hydrophilic matrix formed upon exposure to an environmental fluid. In certain preferred embodiments of the present invention, the hydrophobic material may be included in the sustained release excipient in an amount from about 1% to about 20% by weight of the sustained release excipient. More preferably, the hydrophobic material may be included in the sustained release excipient in an amount from about 1% to about 10%, and most preferably from about 1% to about 5%, by weight of the final formulation. The hydrophobic material may be dissolved in an organic solvent or dispersed in an aqueous solution for incorporation into the formulation.

Preferably, the controlled release excipients of the invention have uniform packing characteristics over a range of different particle size distributions and are capable of processing into tablets using either direct compression, following addition of drug and lubricant powder, conventional wet granulation, or spray granulation techniques.

In certain embodiments, the properties and characteristics of a specific excipient system prepared according to the present invention is dependent in part on the individual characteristics of the homo- and heteropolysaccharide constituents, in terms of polymer solubility, glass transition temperatures etc., as well as on the synergism both between different homo- and heteropolysaccharides and between the homo and heteropolysaccharides and the inert saccharide constituent(s) in modifying dissolution fluid-excipient interactions.

In certain embodiments, the oral dosage form of the present invention may be prepared as granules, spheroids, matrix multiparticulates, etc. which comprise venlafaxine, active metabolite or salt thereof in a sustained release matrix, which may be compressed into a tablet or encapsulated.

In certain embodiments, the complete mixture is in an amount sufficient to make a uniform batch of tablets and is subjected to tableting in a conventional production scale tableting machine at normal compression pressure, i.e. about 2000-1600 lbs/sq in. However, the mixture should not be compressed to such a degree that there is subsequent difficulty in achieving hydration when exposed to gastric fluid. The average tablet weight may be from about 100 mg to 1500 mg.

In certain embodiments of the present invention, the granules, spheroids, matrix multiparticulates, or tableted formulation are coated with a coating layer which may be comprised of a polymer, mixture of polymers, synthetic and/or naturally occurring, that are freely permeable, slightly permeable, water soluble, water insoluble, and polymers whose permeability and/or solubility is affected by pH.

The coating can comprise a hydrophobic material such as those described above. For example, the hydrophobic material may be a hydrophobic polymer, acrylic and/or methacrylic acid polymers or copolymers, hydrogenated vegetable oils, zein, mixtures thereof, as well as other pharmaceutically acceptable hydrophobic materials known to those skilled in the art. Hydrophobic cellulosic materials such as alkyl celluloses may also be used. In certain embodiments the hydrophobic material in the coating is in an amount of from about 2% to about 15% by weight of the final formulation, preferably from about 2% to about 10% by weight of the final formulation. An especially preferred hydrophobic material is ethylcellulose. Ethylcellulose is commercially available as Aquacoat® (aqueous dispersion of ethylcellulose available from FMC) and Surelease® (aqueous dispersion of ethylcellulose available form Colorcon). In certain preferred embodiments, the ethylcellulose (e.g., aqueous dispersion of ethylcellulose) is mixed with a hydrophilic coating material such a hydroxypropylmethylcellulose (commercially available as Opadry® commercially available from Colorcon, West Point, Pennsylvania) prior to coating the final dosage form.

In other embodiments of the present invention, the hydrophobic material is a pharmaceutically acceptable acrylic polymer, including but not limited to acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), glycidyl methacrylate copolymers, and mixtures thereof.

In certain embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In order to obtain a desirable dissolution profile, it may be necessary to incorporate two or more ammonio methacrylate copolymers having differing physical properties, such as different molar ratios of the quaternary ammonium groups to the neutral (meth)acrylic esters. [0203] Certain methacrylic acid ester-type polymers are useful for preparing pH-dependent coatings which may be used in accordance with the present invention. For example, there are a family of copolymers synthesized from diethylaminoethyl methacrylate and other neutral methacrylic esters, also known as methacrylic acid copolymer or polymeric methacrylates, commercially available as Eudragit® from Rohm Pharma.

In certain embodiments, a combination of any of the aforementioned hydrophobic materials may be used.

In embodiments of the present invention where the coating comprises an aqueous dispersion of a hydrophobic material, the inclusion of an effective amount of a plasticizer in the aqueous dispersion of hydrophobic material will further improve the physical properties of the sustained release coating.

Examples of suitable plasticizers for ethylcellulose include water insoluble plasticizers such as dibutyl sebacate, diethyl phthalate, triethyl citrate, tributyl citrate, and triacetin, although it is possible that other water-insoluble plasticizers (such as acetylated monoglycerides, phthalate esters, castor oil, etc.) may be used. Triethyl citrate is an especially preferred plasticizer for the aqueous dispersions of ethyl cellulose of the present invention.

Examples of suitable plasticizers for the acrylic polymers of the present invention include, but are not limited to citric acid esters such as triethyl citrate NF XVI, tributyl citrate, dibutyl phthalate, and possibly 1,2-propylene glycol. Other plasticizers which have proved to be suitable for enhancing the elasticity of the films formed from acrylic films such as Eudragit® RL/RS lacquer solutions include polyethylene glycols, propylene glycol, diethyl phthalate, castor oil, and triacetin. Triethyl citrate is also a preferred plasticizer for the acrylic polymers of the present invention.

Such suitable polymers for inclusion into the coating layer preferably slow the release profile of the dosage form.

In other embodiments of the present invention, the coating layer may comprise an enteric coating material in addition to or instead of the hydrophobic polymer coating. Examples of suitable enteric polymers include cellulose acetate phthalate, hydroxyl propylmethylcellulose phthalate, polyvinylacetate phthalate, methacrylic acid copolymer, shellac, hydroxypropylmethylcellulose succinate, cellulose acetate trimellitate, and mixtures of any of the foregoing. An example of a suitable commercially available enteric material is available under the trade name Eudragit™ L30D55.

In further embodiments, the dosage form may be coated with a hydrophilic coating in addition to or instead of the above-mentioned coatings. An example of a suitable material which may be used for such a hydrophilic coating is hydroxypropylmethylcellulose (e.g., Opadry® as described above).

The coating layer may be applied in any pharmaceutically acceptable manner known to those skilled in the art. For example, in one embodiment, the coating is applied via a fluidized bed or in a coating pan. For example, the coated tablets may be dried, e.g., at about 60-70° C. for about 3-4 hours in a coating pan. The solvent for the hydrophobic polymer or enteric coating may be organic, aqueous, or a mixture of an organic and an aqueous solvent. The organic solvents may be, e.g., isopropyl alcohol, ethanol, and the like, with or without water.

In additional embodiments of the present invention, a support platform is applied to the tablets manufactured in accordance with the present invention. Suitable support platforms are well known to those skilled in the art. An example of suitable support platform is set forth, e.g., in U.S. Pat. No. 4,839,177, hereby incorporated by reference. In that patent, the support platform partially coats the tablet, and consists of a polymeric material insoluble in aqueous liquids. The support platform may, for example, be designed to maintain its impermeability characteristics during the transfer of the therapeutically active medicament. The support platform may be applied to the tablets, e.g., via compression coating onto part of the tablet surface, by spray coating the polymeric materials comprising the support platform onto all or part of the tablet surface, or by immersing the tablets in a solution of the polymeric materials.

The support platform may have a thickness of, e.g., about 2 mm if applied by compression, and about 10 μm if applied via spray-coating or immersion-coating.

Generally, in embodiments of the invention wherein a coating comprising a hydrophobic material or enteric coating material is applied to a core, the cores are coated to a weight gain from about 1 to about 20%, and in certain embodiments preferably from about 5% to about 15%, in certain preferred embodiments from about 7% to about 15%, and in a particular preferred embodiment, about 11%. In certain embodiments, the coating comprising the hydrophobic material is in an amount of from about 1% to about 20, preferably from about 2% to about 15% by weight of the final formulation.

Additionally, the cores may optionally be coated with a color coat that rapidly disintegrates or dissolves in water or the environment of use. The color coat may be a conventional sugar or polymeric film coating which is applied in a coating pan or by conventional spraying techniques. Preferred materials for the color coat are commercially available under the Opadry tradename (e.g, Opadry® II White, Opadry® II Blue). The color coat may be applied directly onto the tablet core, or may be applied after a coating as described above. Generally, the color coat surrounding the core will comprise from about 1 to 5% preferably about 2 to 4% based on the total weight of the tablet.

An effective amount of any generally accepted pharmaceutical lubricant or mixture of lubricants, including the calcium or magnesium soaps may be added to the above-mentioned ingredients of the formulation at the time the medicament is added, or in any event prior to compression into a solid dosage form. Preferably the lubricant is in an amount of from about 0.5% to about 10%, more preferably from about 0.5% to about 5% by weight of the final formulation. An example of a suitable lubricant is magnesium stearate in an amount of about 0.5% to about 3% by weight of the solid dosage form. An especially preferred lubricant is sodium stearyl fumarate, NF, commercially available under the trade name Pruv® from JRS Pharma LP. Another preferred lubricant is talc.

An effective amount of any generally acceptable pharmaceutical glidant or mixture of glidants may also be added to the above-mentioned ingredients of the formulation at the time the medicament is added, or in any event prior to compression into a solid dosage form, including colloidal silicon dioxide, talc, silicon dioxide, sodium aluminosilicate, calcium silicate, powdered cellulose, microcrystalline cellulose, corn starch, sodium benzoate, calcium carbonate, magnesium carbonate, metallic stearates, calcium stearate, magnesium stearate, zinc stearate, stearowet C, starch, starch 1500, magnesium lauryl sulfate, and magnesium oxide. In certain embodiments, a glidant may also be added to the material to be coated prior to application. Preferably the glidant is in an amount of from about 0.5% to about 10%, preferably from about 2% to about 8% by weight of the final formulation.

In certain embodiments, defoaming agents, also known as antifoaming agents, are included in the dosage forms of the present invention. The antifoaming agents are substances used to reduce foaming due to mechanical agitation or to gases, nitrogenous materials or other substances which may interfere during processing. Examples include metallic salts such as sodium chloride; C₆ to C₁₂ alcohols such as octanol; sulfonated oils; silicone ethers such as simethicone; organic phosphates and the like. The amount of antifoaming agent in the composition can range from about 0.005 to about 5%, preferably from about 0.01 to about 2%.

In certain embodiments, additional inert diluents may also be incorporated in the sustained release oral dosage form when mixing the sustained release excipient with the venlafaxine, an active metabolite of venlafaxine or a pharmaceutically acceptable salt thereof. The inert diluent may be the same or different inert diluent that is incorporated into the sustained release excipient. Other pharmaceutically acceptable diluents and excipients that may be used to formulate oral dosage forms of the present invention are described in the Handbook of Pharmaceutical Excipients, American Pharmaceutical Association (1986).

In certain embodiments, the controlled release excipients of the present invention are prepared via a wet granulation method. However, the controlled release excipients prepared in accordance with the present invention may be prepared according to any agglomeration technique to yield an acceptable excipient product. In wet granulation techniques, for example, the desired amounts of the heterpolysaccharide gum, the homopolysaccharide gum, optional cationic cross-linking agent and the inert diluent are mixed together and thereafter a moistening agent such as water, propylene glycol, glycerol, alcohol or the like is added to prepare a moistened mass. Next, the moistened mass is dried. The dried mass is then milled with conventional equipment to obtain the desired particle size.

Once the sustained release excipient of the present invention has been prepared, it is then possible to blend the same with the venlafaxine, active metabolite or salt thereof, e.g., in a V-blender and compress the blend into an extended release oral tablet.

In certain preferred embodiments, the mixture of sustained release excipient and the active ingredient e.g., venlafaxine hydrochloride (and optionally additional diluent and excipients) may be spray granulated with a solution or suspension of e.g., a cellulose derivative such as an alkylcellulose, hydroxyalkylcellulose, hydroxyalkylalkylcellulose, or mixtures thereof. Preferably, the cellulose derivative is an alkylcellulose such as ethylcellulose, methylcellulose, and the like; a hydroxylalkylcellulose such as hydroxyethylcellulose, hydroxypropylcellulose, and the like; a hydroxylalkylalkylcellulose such as hydroxypropylmethylcellulose, hydroxyethylmethylcellulose, and the like; or mixtures thereof. In certain alternate embodiments, the sustained release excipient may be spray granulated with the cellulose derivative prior to incorporation of the active ingredient, e.g., venlafaxine hydrochloride. Preferably the cellulose derivative used in the spray granulation (e.g., hydroxypropylmethylcellulose) is in the final formulation in an amount of from about 1% to about 10%, preferably from about 2 to about 6% by weight of the final formulation. Preferably the inclusion of the cellulose derivative via spray granulation aids the processing (e.g., tableting) of the formulations (e.g., decreases sticking of granulated powders to the tablet press).

Preferably the granules are compressed into tablets. Although tablets are preferred dosage forms of the present invention, the ingredients may also be formulated in a capsule, extruded and spheronized with an active medicament to form pellets, etc.

In certain preferred embodiments, after the granules are compressed into tablets, the tablets are overcoated with one or more of the coatings described above. In certain embodiments, however, the matrix is capable of providing the desired controlled release without a controlled or delayed release coating.

In tablet formulations, the tablets have a hardness from about 7 to about 20 kp.

### DETAILED DESCRIPTION OF PREFERRED EMBODIMENTS

The following examples illustrate various aspects of the present invention. They are not to be construed to limit the claims in any manner whatsoever.

### Examples 1 to 7 are compositions of Venlafaxine HCl ER Tablets

Examples 1, 2, 5 & 6 are examples that describe the effect of amount of gum, Examples 3-5 are examples that describe pseudo-dose proportionality, and examples 2, 6 & 7 are Examples that describe dose-proportionality.

### EXAMPLES 1-7

In Examples 1-7, a sustained release excipient (70% gum) in accordance with the present invention was prepared. The sustained release excipient was prepared by dry blending the requisite amounts of xanthan gum, locust bean gum, calcium sulfate and mannitol in a high speed mixer/granulator. While running choppers/impellers, water was added to the dry blended mixture, and granulated. The granulation was then dried in a fluid bed dryer to a LOD (loss on drying) of less than about 10% by weight (e.g., 4-7% LOD). The granulation was then milled using comminuting machine. The ingredients of the sustained release excipient of Examples 1-7 are listed in Table I below:

TABLE 1

| Component | Amount (%) |
|---|---|
| Xanthan Gum | 28% |
| Locust Bean Gum | 42% |
| Calcium Sulfate Dihydrate | 10% |
| Mannitol, USP | 20% |
| Water* | q.s. |

*Removed during processing

To study the effect of gum, dose-proportionality and pseudo-dose proportionality, different percentages of sustained release excipient from Examples 1-7 prepared as described above were dry blended with a desired amount of venlafaxine. The prepared formulations of Examples 1-7 are set forth below in Table 2:

TABLE 2

| | Example 1 | Example 2 | Example 3 | Example 4 | Example 5 | Example 6 | Example 7 |
|---|---|---|---|---|---|---|---|
| Ingredient | mg/tab | mg/tab | mg/tab | mg/tab | mg/tab | mg/tab | mg/tab |
| Sustained | 638.2 | 450 | 450 | 450 | 450 | 112.5 | 225 |
| Release | (79.8%) | (73.8%) | (74.6%) | (85.3%) | (91.8%) | (73.8%) | (73.8%) |
| Excipient (70%) | | | | | | | |
| Venlafaxine | 150 | 150 | 150 | 75 | 37.5 | 37.5 | 75 |
| HCl | (18.8%) | (24.6%) | (24.9%) | (14.2%) | (7.7%) | (24.6%) | (24.6%) |
| Sodium stearyl | 11.8 | 10 | 3 | 2.6 | 2.5 | 2.5 | 5 |
| fumarate | (1.5%) | (1.6%) | (0.5%) | (0.5%) | (0.5%) | (1.6%) | (1.6%) |
| Total Weight | 800 | 610 | 603 | 527.6 | 490 | 152.5 | 305 |
| Active:Gum | 1:2.98 | 1:2.1 | 1:2.1 | 1:4.2 | 1:8.4 | 1:2.1 | 1:2.1 |
| Tooling Size | 0.280″ × | 0.2500″ × | 0.2500″ × | 0.2500″ × | 0.2500″ × | 9/32″ | 5/16″ |
| | 0.750″ | 0.7250″ | 0.7250″ | 0.6750″ | 0.6750″ | | |

NOTE:
Range of active:Gum used in Examples 1-7 is 1:2.1 to 1:8.4.

The formulations of Examples 1-7 were prepared as follows:
1. Venlafaxine HCl is sifted through a sieve and mixed with the sustained release excipient in a high shear granulator.
2. The dry blend of step 1 is granulated with water until consistent granulation is achieved.
3. The wet mass of step 2 is dried in fluidized bed dryer.
4. dried granules of step 3 is passed through a Fitzmill.

5. Sodium stearyl fumarate is passed through a sieve and mixed with the milled granules of step 4.

6. The lubricated blend of step 5 is compressed to make the tablets of about 800 mg for Example 1 and 610 mg for Example 2.

The tablets prepared in accordance with Examples 1-7 were dissolution tested in USP dissolution Apparatus type III, in pH change (pH of 1.5 for the first hour, with a switch to pH 4.5 for two hours, with a switch to pH 7.5 thereafter) with an agitation of 15 dpm. The volume and temperature for the media were 250 ml and 37° C., respectively. The tablets were tested at 0, 2, 4, 8, 16, and 24 hour time points. The dissolution results are listed in Table 3 below:

TABLE 3

| Dissolution time | | | | | | | |
|---|---|---|---|---|---|---|---|
| (hours) | Example 1 | Example 2 | Example 3 | Example 4 | Example 5 | Example 6 | Example 7 |
| 0 | 0 | 0 | 0 | 0 | 0 | 0 | 0 |
| 1 | 23.2 | 28.3 | 29.6 | 28.1 | 29.6 | 41.0 | 31.9 |
| 2 | 33.3 | 40.3 | 38.9 | 39.6 | 41.5 | 58.9 | 45.6 |
| 4 | 47.7 | 56.6 | 57.3 | 56.7 | 59.0 | 78.6 | 63.2 |
| 8 | 66.7 | 76.7 | 81.6 | 80.2 | 83.8 | 93.5 | 82.5 |
| 16 | 86.7 | 92.1 | 97.3 | 96.8 | 99.7 | 99.2 | 96.4 |
| 24 | 95.6 | 96.4 | 98.9 | 98.7 | 100.9 | 100.0 | 99.7 |
| Remnant | 3.2 | 0.4 | 0.0 | 0.0 | 0.0 | 0.0 | 0.3 |
| % Recovery | 98.8 | 96.8 | 98.9 | 98.7 | 100.9 | 100.0 | 100.0 |

### Examples 8 to 14 are Compositions of Venlafaxine HCl ER Tablets

To study the effect of extra-granular sustained release excipient and grades of sustained release excipient, Examples 8 to 12 and Examples 13 and 14 were prepared in accordance with the present invention. Examples 8 to 12 illustrate the effect of extra-granular sustained release formulations and Examples 13 to 14 are Examples that illustrate the effect of grades of sustained release excipients in accordance with the present invention. The ingredients of the sustained release excipient (70% gum) of Examples 8-13 are the same as the ingredients listed in Table I above, for Examples 1-7.

The ingredients of the sustained release excipient (50% gum) of Example 14 are the set forth in Table 4:

TABLE 4

| Component | Amount (%) |
|---|---|
| Xanthan Gum | 20% |
| Locust Bean Gum | 30% |
| Calcium Sulfate Dihydrate | 10% |
| Mannitol, USP | 40% |
| Water* | q.s. |

*Removed during processing

The prepared formulations of Examples 8 to 14 are listed below in Table 5:

TABLE 5

| Ingredient | Example 8 | Example 9 | Example 10 | Example 11 | Example 12 | Example 13 | Example 14 |
|---|---|---|---|---|---|---|---|
| Part A | mg/tab | mg/tab | mg/tab | mg/tab | mg/tab | mg/tab | mg/tab |
| Sustained release | 450 (62.1%) | 450 (44.3%) | 642.5 (64.2%) | 450 (54.6) | 450 (45%) | 638 (78.2%) | — |
| excipient-(70%) | | | | | | | |
| Sustained release | — | — | — | — | — | — | 900 (83.3%) |
| excipient (50%) | | | | | | | |
| Venlafaxine HCl | 170 (23.1%) | 170 (16.7%) | 170 (17%) | 170 (20.7%) | 170 (17%) | 170 (20.8%) | 170 (15.7%) |
| 25% Surelease E-7- | 113.3 (15.4%) | 113.3 (11.2%) | — | — | — | — | — |
| 7050 dispersion* | | | | | | | |
| Hypromellose, USP | — | — | — | — | 50 (5%) | — | — |
| Silicon dioxide, NF | — | — | — | — | — | — | 5 (0.5%) |
| Magnesium Stearate, | — | — | 4.1 (0.4%) | — | — | 8.0 (1.0%) | 5 (0.5%) |
| NF | | | | | | | |
| Part B | — | — | — | — | — | — | — |
| Sustained release | — | 267.7 (27.3%) | 182.5 (18.3%) | 200 (24.3%) | 235 (23.5% | — | — |
| excipient (70%) | | | | | | | |
| Prosolv 90 | — | — | — | — | 90 (9%) | — | — |
| Magnesium Stearate, | 3.1 (0.4%) | 5.0 (0.5%) | 0.9 (0.1%) | 3.1 (0.4%) | 5 (0.5% | — | — |
| NF | | | | | | | |
| Total Weight | 736.4 | 1015.0 | 1000.0 | 823.1 | 1000 | 816 | 1080 |
| Active:Gum | 1:1.85 | 1:2.74 | 1:3.4 | 1:2.67 | 1:2.8 | 1:2.63 | 1:2.65 |
| Tooling Size | 0.3125″ × | 0.3125″ × | N/A | 0.3125″ × | 0.3125″ × | 0.345″ × | 0.345″ × |
| | 0.7500″ | 0.7500″ | | 0.7500″ | 0.7500″ | 0.700″ | 0.700″ |
| Hardness (Kp) | ˜14 | ˜20 | — | ˜10 | ˜19 | ˜7.0 | ˜12 |
| Friability (%) | — | — | — | — | 0.06 | 1.7 | 0.37 |

*as dry ethylcellulose material
NOTE:
Range of Active:Gum used in Examples 8-14 is 1:1.85 to 1:3.4.

### EXAMPLE 8

The formulation of Example 8 was prepared as follows:
1. A sustained release excipient (70% gum) is prepared in accordance with Examples 1-7.
2. Venlafaxine HCL is sifted through a sieve and mixed with the sustained release excipient in a Fluid Bed Granulator/Drier.
3. Add the required amount of water to 25% Surelease to achieve a 20% dispersion of Surelease and stir for approximately 30 minutes.
4. The dry blend of step 2 is granulated with the granulation dispersion of step 3 in a fluid bed granulator.
5. The granules of step 4 are dried in the fluid bed dryer to a moisture content of less than 3.5%.
6. The dried granules of step 5 are sifted through a sieve and charged into a V-Blender.
7. Magnesium stearate is passed through a sieve and mixed with the mixture of step 6.
8. The lubricated blend of step 7 is compressed to make tablets of about 620 mg.

### EXAMPLE 9

The formulation of Example 9 was prepared as follows:
1. A sustained release excipient (70% gum) is prepared in accordance with Examples 1-7.
2. Venlafaxine HCL is sifted through a sieve and mixed with the sustained release excipient in a Fluid Bed Granulator.
3. Add the required amount of water to 25% Surelease to achieve a 20% dispersion of Surelease and stir for approximately 30 minutes.
4. The dry blend of step 2 is granulated with the granulation dispersion of step 3 in a fluid bed granulator.
5. The granules of step 4 are dried in the fluid bed dryer to a moisture content of less than 3.5%.
6. The dried granules of step 5 are sifted through a sieve and charged into a V-Blender.
7. A second sustained release excipient (comprising the same components of the excipient of step 1) is added to the V-Blender of step 6 and mixed.
8. Magnesium stearate is passed through a sieve and mixed with the mixture of step 7 in the V-Blender.
9. The lubricated blend of step 8 is compressed to make tablets of about 1015 mg.

### EXAMPLE 10

The formulation of Example 10 was prepared as follows:
1. A sustained release excipient (70% gum) is prepared in accordance with Examples 1-7.
2. Venlafaxine HCL is sifted through a sieve and mixed with the sustained release excipient in a high shear granulator.
3. The dry blend of step 2 is granulated with water in the high shear granulator.
4. The granules of step 3 are dried in the fluid bed dryer to a moisture content of less than 3.5%.
5. The dried granules of step 4 are sifted through a sieve and charged into a V-Blender.
6. Magnesium stearate is passed through a sieve and mixed with the mixture of step 5 in the V-Blender.
7. A second sustained release excipient and second sieved magnesium stearate are blended separately and tablets are compressed with the blend of step 5 and step 6 forming tablets of about 1000 mg.

### EXAMPLE 11

The formulation of Example 11 was prepared as follows:
1. A sustained release excipient (70% gum) is prepared in accordance with Examples 1-7.
2. Venlafaxine HCL is sifted through a sieve and mixed with the sustained release excipient in a high shear granulator.
3. The dry blend of step 2 is granulated with water in the high shear granulator.
4. The granules of step 3 are dried in the fluid bed dryer to a moisture content of less than 3.5%.
5. The dried granules of step 4 are sifted through a sieve and charged into a V-Blender.
6. A second sustained release excipient (comprising the same components of the excipient of step 1) is added to the V-Blender of step 5 and mixed.
7. Magnesium stearate is passed through a sieve and mixed with the mixture of step 6 in the V-Blender.
8. The lubricated blend of step 7 is compressed to make tablets of about 823mg.

### EXAMPLE 12

The formulation of Example 12 was prepared as follows:
1. A sustained release excipient (70% gum) is prepared in accordance with Examples 1-7.
2. Venlafaxine HCL is sifted through a sieve and mixed with the sustained release excipient in a Fluid Bed Granulator.
3. The dry blend of step 2 is granulated with 10%HPMC solution in the high shear granulator.
4. The granules of step 4 are dried in the fluid bed dryer to a moisture content of less than 3.5%.
5. The dried granules of step 4 are sifted through a sieve and charged into a V-Blender.
6. A second sustained release excipient (comprising the same components of the excipient of step 1) is added to the V-Blender of step 5 and mixed.
7. Prosolv 90 is mixed with the mix of step 6 in the V-Blender.
8. Magnesium stearate is passed through a sieve and mixed with the mixture of step 7 in the V-Blender.
9. The lubricated blend of step 8 is compressed to make tablets of about 1000 mg.

### EXAMPLE 13

The formulation of Example 13 was prepared as follows:
1. A sustained release excipient (70% gum) is prepared in accordance with Examples 1-7.
2. Venlafaxine HCL is sifted through a sieve and mixed with the sustained release excipient in a high shear granulator.
3. The dry blend of step 2 is granulated with water in the high shear granulator.
4. The granules of step 3 are dried in the fluid bed dryer to a moisture content of less than 3.5%.
5. The dried granules of step 4 are sifted through a sieve and charged into a V-Blender.
6. Magnesium stearate is passed through a sieve and mixed with the mixture of step 5 in the V-Blender.
7. The lubricated blend of step 6 is compressed to make tablets of about 816mg.

### EXAMPLE 14

The formulation of Example 14 was prepared as follows:
1. A sustained release excipient (50% gum) is prepared in accordance with process of Examples 1-7, but using the ingredients listed in Table 4.
2. Venlafaxine HCL is sifted through a sieve and mixed with the sustained release excipient in a high shear granulator.
3. The dry blend of step 2 is granulated with water in the high shear granulator.
4. The granules of step 3 are dried in the fluid bed dryer to a moisture content of less than 3.5%.
5. The dried granules of step 4 are sifted through a sieve and charged into a V-Blender.
6. Silicon dioxide and magnesium stearate are passed through a sieve and mixed with the milled granules of step 5 in the V-Blender.
7. The lubricated blend of step 6 is compressed to make tablets of about 1080 mg.

The tablets prepared in accordance with Examples 1-7 were dissolution tested in USP dissoluation Apparatus type III, with media pH change at an agitation of 15 dpm. The volume and temperature for the media were 250 ml and 37° C., respectively. The tablets were tested at 0, 2, 4, 8, 16, and 24 hour time points. The dissolution results are listed in Table 6 below:

TABLE 6

| Dissolution | | | | | | | |
|---|---|---|---|---|---|---|---|
| time (hours) | Example 8 | Example 9 | Example 10 | Example 11 | Example 12 | Example 13 | Example 14 |
| 0 | 0 | 0 | 0 | 0 | 0 | 0 | 0 |
| 1 | 26.7 | 21.3 | 15.6 | 21.1 | 24.4 | 25.1 | 25.2 |
| 2 | 37.4 | 29.8 | 22.1 | 30.3 | 33.8 | 35.9 | 36.3 |
| 4 | 51.0 | 40.1 | 33.3 | 45.1 | 47.4 | 50.6 | 51.7 |
| 8 | 71.9 | 58.8 | 54.6 | 68.6 | 67.9 | 72.1 | 74.8 |
| 16 | 92.9 | 80.3 | 81.2 | 95.6 | 92.1 | 92.0 | 94.6 |
| 24 | 99.4 | 90.7 | 93.4 | 100.9 | 99.1 | 94.8 | 97.9 |
| Remnant | 0.8 | 4.5 | 4.3 | 1.9 | 0.4 | 0.1 | 0.2 |
| % Recovery | 100.2 | 95.2 | 97.7 | 101.0 | 99.5 | 94.9 | 98.1 |

### EXAMPLES 15 TO 18

Examples 15 and 16 are examples that describe the effect of binder. Examples 15 and 17 are examples that describe the effect of amount of binder. Examples 17 and 18 are examples that describe the effect of the process of manufacture.

The formulations of Examples 15 to 18 are listed in Table 7 below:

TABLE 7

| | Example 15 | Example 16 | Example 17 | Example 18 |
|---|---|---|---|---|
| Ingredient | mg/tab | mg/tab | mg/tab | mg/tab |
| Sustained release | 638 (67.2%) | 638 (66%) | 638 (62%) | 638 (68.2%) |
| excipient (70% gum) | | | | |
| Venlafaxine HCl | 170 (17.9%) | 170 (17.6%) | 170 (16.5%) | 170 (18.2%) |
| Hypromellose, USP | — | 60.5 (6.3%) | — | — |
| 25% Surelease E-7-7050 | 50 (5.3%) | — | 120 (11.2%) | 120 (12.8%) |
| dispersion* | | | | |
| Prosolv 90 | 86 (9.1%) | 93 (9.6%) | 93 (9%) | — |
| Magnesium Stearate, NF | 5 (0.5%) | 5 (0.5%) | 8 (0.8%) | 8 (0.9%) |
| Total Weight | 949 | 967 | 1029 | 936 |
| Active:Gum | 1:2.63 | 1:2.63 | 1:2.63 | 1:2.63 |
| Tooling Size | 0.345″ × 0.700″ | 0.345″ × 0.700″ | 0.345″ × 0.700″ | 0.345″ × 0.700″ |
| Process | Spray | Spray | Spray | Wet granulation |
| | granulation | granulation | granulation | |
| Hardness (Kp) | ˜12-14 | ˜11-13 | ˜19-20 | ˜9-11 |
| Friability (%) | 0.18 | 0.16 | 0.0 | 0.03 |

*as dry ethylcellulose material

### EXAMPLE 15

The formulation of Example 15 was prepared as follows:
1. Venlafaxine HCl is sifted through sieve and mixed with the sustained release excipient (70% gum) of Examples 1-7, in a fluid bed granulator/drier.
2. Add the required amount of water to 25% Surelease to achieve a 20% dispersion of Surelease and stir for approximately 30 minutes.
3. Dry blend of step 1 is granulated with the granulation dispersion as specified in step 2 in the Fluid Bed Granulator/Drier.
4. The granules of step 3 are dried in the fluid bed dryer to a moisture content of less than 3.5%.
5. Prosolv 90 is mixed with the sieved dried granules of step 4 in a V-Blender.
6. Magnesium stearate is passed through a sieve and mixed with the mix of step 5.
7. Lubricated blend of step 6 is compressed to make tablets of about 949 mg.

### EXAMPLE 16

The formulation of Example 16 was prepared in accordance with the process of Example 15 except the lubricated blend of step 6 was compressed to make tablets of about 967 mg.

### EXAMPLE 17

The formulation of Example 17 was prepared in accordance with the process of Example 15 except the lubricated blend of step 6 was compressed to make tablets of about 1029 mg.

### EXAMPLE 18

The formulation of Example 18 was prepared as follows:
1. Venlafaxine HCl is sifted through sieve and mixed with sustained release excipient (70% gum) of Examples 1-7, in a high shear granulator.
2. Dry blend of step I is granulated with 25% Surelease dispersion in the high shear granulator and water is added when needed.
3. The granules of step 2 are dried in the high shear granulator to a moisture content of less than 3.5%.
4. Magnesium stearate is passed through a sieve and mixed with the mix of step 3 in
5. The lubricated blend of step 4 is compressed to make the tablets of about 936 mg.

The tablets prepared in accordance with Examples 1-7 were dissolution tested in USP dissolution Apparatus type III, with media pH change at an agitation of 15 dpm. The volume and temperature for the media were 250 ml and 37° C., respectively. The tablets were tested at 0, 2, 4, 8, 16, and 24 hour time points. The dissolution results are listed in Table 8 below:

TABLE 8

| Dissolution | | | | |
|---|---|---|---|---|
| time (hours) | Example 15 | Example 16 | Example 17 | Example 18 |
| 0 | 0 | 0 | 0 | 0 |
| 1 | 24.6 | 25.6 | 24.2 | 21.2 |
| 2 | 34.4 | 35.7 | 34.0 | 30.2 |
| 4 | 47.5 | 50.3 | 46.2 | 41.5 |
| 8 | 67.7 | 74.3 | 65.0 | 59.3 |
| 16 | 89.4 | 96.6 | 89.7 | 82.4 |
| 24 | 96.0 | 101.8 | 99.0 | 92.1 |
| Remnant | 1.3 | 0.5 | 1.9 | 3.4 |
| % Recovery | 97.3 | 102.3 | 101.9 | 95.5 |

### EXAMPLES 19-21

Formulations containing 170 mg and 85 mg of Venlafaxine HCl that are equivalent to 150 mg and 75 mg of venlafaxine base, respectively were evaluated. The formulations were prepared by spray granulation, using different gum ratios. Example 19 had a drug:gum ratio of 1:2.627. The resulting release rate profiles were evaluated under USP III Method in 250 ml at a pH of 7.5 at 37° C., 15dpm, tested at 0, 1, 2, 4, 8, 16, and 24 hour time points.

The sustained release excipient of Examples 19-21 had the same ingredients as the excipient of Table 1 and was prepared in accordance with the process of Examples 1-7.

### Composition of 170 mg and 85 mg of Venlafaxine HCl ER Formulations

TABLE 9

| | Example 19 | Example 20 | Example 21 |
|---|---|---|---|
| Ingredient | Amount in mg | Amount in mg | Amount in mg |
| Venlafaxine HCl | 170.0 | 170.0 | 85.0 |
| Sustained release excipient | 638.0 | 300.0 | 319.0 |
| (70% gum) | | | |
| 25% Surelease ® E-7-7050 dispersion | 120.0 | 70.0 | 60.0 |
| (as dry ethylcellulose material) | | | |
| Prosolv 90 ® | 93.0 | 54.0 | 46.5 |
| Magnesium Stearate, NF | 8.0 | 3.0 | 4.0 |
| Sterile Water For Injection, USP | * | * | * |
| Process | Spray Granulation | Spray Granulation | Spray Granulation |
| Active to Gum Ratio | 1:2.627 | 1:1.2 | 1:2.627 |
| Total Weight | 1029.0 | 597.0 | 514.5 |
| Hardness | 14-16 Kp | 16-19 | N/A |
| Tooling Size | 0.3450″-0.7000″ | 0.2800″ × 0.6000″ | — |

*Removed during processing

The formulations of Examples 19-21 were prepared as follows:
1. Weigh Venafaxine HCl and pass through a No.20 mesh screen.
2. While stirring, add water to 25% Surelease E-7-7050 dispersion to prepare a 20% Surelease E-7-7050 dispersion.
3. Charge the sustained release excipient (70%) and Venlafaxine HCl into the UniGlatt Fluid Bed Processor and pre-blend the mixture.
4. Spray granulate the 20% Surelease dispersion into the mixture from Step 3 in the UnitGlatt Fluid Bed processor.
5. Dry the granules in the UniGlatt Fluid Bed Processor until 3.5% LOD is achieved.
6. Screen the dried granules from Step 5 through #20 mesh screen.
7. Weigh Prosolv 90 and magnesium stearate, dry blend using a Patterson Kelly Blend master V-Blender with the screened granules.
10. Compress blend into tablets in a Cadmach using 0.3450 - 0.7000 inch modified oval shaped punches.

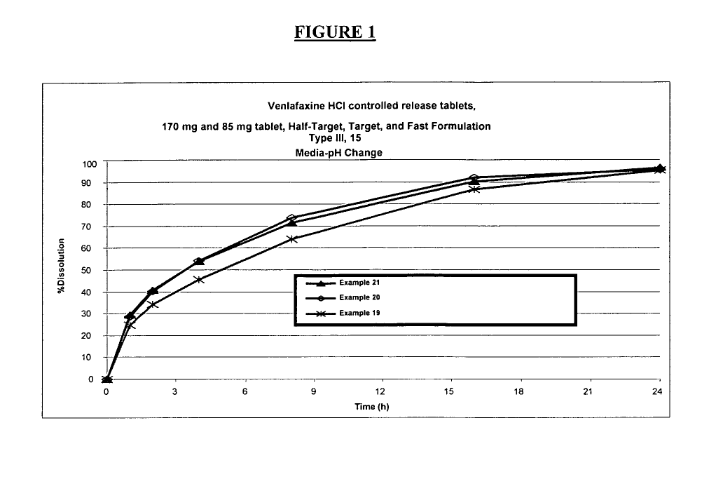

The tablets prepared in accordance with Examples 19 to 21 were dissolution tested in USP dissolution Apparatus type III, at pH change media (pH of 1.5 for the first hour, with a switch to pH 4.5 for two hours, with a switch to pH 7.5 thereafter) with an agitation of 15 dpm. The volume and temperature for the media were 250 ml and 37° C., respectively. The tablets were tested at 0, 1, 2, 4, 8, 16, and 24 hour time points. The dissolution results are shown in FIG. 1. The dissolution results are listed in table 10 below:

TABLE 10

| | Example 19 | Example 20 | Example 21 |
|---|---|---|---|
| Time | (170 mg, | (170 mg, | (½ × 170 mg = 85 mg), |
| (h) | 1:2.627) | 1:1.2) | 1:2.627) |
| 0 | 0 | 0 | 0 |
| 1 | 24.6 | 28.5 | 29.5 |
| 2 | 34.1 | 40.1 | 40.7 |
| 4 | 45.6 | 54.3 | 53.9 |
| 8 | 63.9 | 73.9 | 71.4 |
| 16 | 86.6 | 91.9 | 90.1 |
| 24 | 95.2 | 95.6 | 96.4 |
| Remnant | 2.8 | 0.8 | 1.7 |
| % Recovery | 97.9 | 96.4 | 98.1 |

The results show that the dissolution rate of the formulation of Example 19, having an active to gum ratio of 1:2.627 was about 10% slower than the rate of the formulation of Example 20, having an active to gum ratio of 1:1.2 at 8 hours. The dissolution rate of the formulation of Example 19 was about 8% slower than the rate of the formulation of Example 21 at 8 hours.

### EXAMPLE 22

In the manufacture of Examples 22A, 22B and 22C, venlafaxine HCl and sustained release excipient of Table 1 (70%) were granulated with Surelease® E-7-7050 dispersion in a Uni-Glatt Fluid Bed Dryer and dried in the same dryer to achieve LOD less than 3.5%. The dried granulations are passed through a Fitzmill Knives forward using Screen #1521-0050 with ˜1585 speed. The milled dried granulation was blended with Prosolv 90M for 10 minutes in a V-blender. Magnesium stearate was added to the mix and blended for 3 minutes in the V-Blender. The final blend was compressed into tablets. All tablet strengths were compressed using 0.2987″×0.5890″. All tablet strengths are scored. The tablets were film coated in a Vector coating pan with ˜3% Opadry II. Different coating colors were used for each of the three strengths. The final formulations are presented in Table 11.

TABLE 11

| | Example | | | | | |
|---|---|---|---|---|---|---|
| | 22a | 22B | 22C | 22a | 22B | 22C |
| Components | Concentration (mg/tablet) | | | % (w/w) | | |
| Strength (mg) | 37.5 | 75 | 150 | 37.5 | 75 | 150 |

Tablet Core
Intra-granular

| | | | | | | |
|---|---|---|---|---|---|---|
| Venlafaxine HCl | 42.5 | 85.0 | 170.0 | 9.05 | 16.60 | 28.48 |
| Sustained release excipient | 300 | 300.0 | 300.0 | 63.90 | 58.59 | 50.25 |
| (70%) | | | | | | |
| Surelease E-7-7050 (Solids) | 70 | 70.0 | 70.0 | 14.91 | 13.67 | 11.73 |

Extra-granular

| | | | | | | |
|---|---|---|---|---|---|---|
| Prosolv 90M | 54 | 54.0 | 54.0 | 11.50 | 10.55 | 9.05 |
| Magnesium Stearate | 3.0 | 3.0 | 3.0 | 0.64 | 0.59 | 0.50 |
| Purified Water, USP* | * | * | * | * | * | * |
| Core Tablet Total | 469.5 | 512.0 | 597.0 | 100 | 100 | 100 |

Color Coating

| | | | | | | |
|---|---|---|---|---|---|---|
| Opadry ® II White 49B18554 | 14.085 | — | | | | |
| Opadry ® II Blue 49B90774 | | 15.36 | | | | |
| Opadry ® II Purple 49B10154 | | | 17.91 | | | |
| Purified water, USP* | * | * | * | * | * | * |
| Coated Tablet Total | 483.585 | 527.36 | 614.91 | | | |

*Removed during processing.

All tablets strengths were prepared from the same components. While not strictly dose proportional, the three formulations contain quantitatively the same amounts of each inactive ingredients used in the core tablet formulations.

### EXAMPLE 23

Example 23 reports comparative dissolution studies that have been completed studying the effect of 40%EtOH/pH 1.5 and 40%EtOH/pH 4.5 in the in-vitro release of Examples 22A-C and Effexor® XR capsules ˜37.5 mg, 75 mg, and 150 mg.

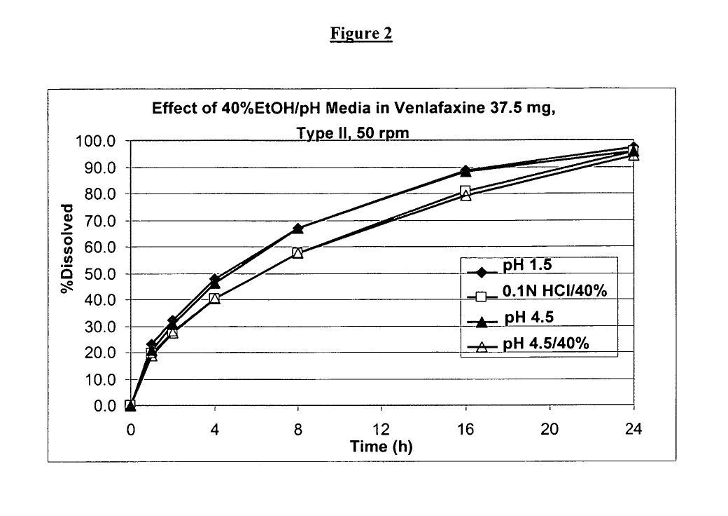

This study was performed with Examples 22A-C and Effexor® XR capsules in pH 1.5; 0.1 N HCl/40%EtOH; pH 4.5; and pH 4.5/40%EtOH. Three samples of Effexor® XR were used for the in-vitro analysis of 37.5 mg, 75 mg, and 150 mg capsules, respectively. The dissolution profiles are shown in FIGS. 2-9.

### EXAMPLE 23A

The formulation of Example 22A was subjected to in-vitro dissolution testing with the following parameters: Apparatus—USP Type 11; Volume—900 ml; Rotational Speed—50 rpm; Media—40% Ethanol in 0.1 N HCl or pH 4.5. The results are set forth in Table 12 and FIG. 2.

TABLE 12

| Time (h) | pH 1.5 | 0.1N HCl/40% | pH 4.5 | pH 4.5/40% |
|---|---|---|---|---|
| 0 | 0.0 | 0.0 | 0.0 | 0.0 |
| 1 | 23.0 | 19.6 | 20.9 | 18.8 |
| 2 | 32.3 | 28.0 | 30.7 | 27.6 |
| 4 | 47.8 | 40.4 | 46.2 | 40.2 |
| 8 | 66.9 | 57.6 | 67.0 | 57.6 |
| 16 | 88.8 | 80.6 | 88.2 | 79.3 |
| 24 | 97.3 | 95.5 | 95.5 | 94.0 |

As seen from the results, the release of Example 22A is about 3-10% slower in 0.1 N HCl and in pH 4.5 with 40%EtOH than it is in the same media without EtOH.

### EXAMPLE 23B

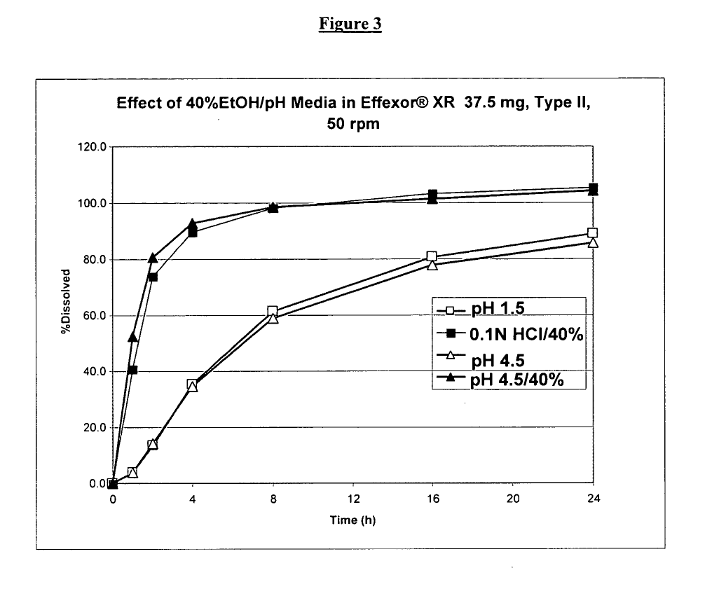

Effexor® XR 37.5 mg Capsules was subjected to in-vitro dissolution testing with the following parameters: Apparatus—USP Type 11; Volume—900 ml; Rotational Speed—50 rpm; Media—40% Ethanol in 0.1 N HCl or pH 4.5. The results are set forth in Table 13 and FIG. 3.

TABLE 13

| Time | | 0.1N HCl/ | | pH 4.5/ |
|---|---|---|---|---|
| (h) | pH 1.5 | 40% EtOH | pH 4.5 | 40% EtOH |
| 0 | 0.0 | 0.0 | 0.0 | 0.0 |
| 1 | 4.0 | 40.7 | 4.1 | 52.4 |
| 2 | 13.7 | 73.9 | 14.2 | 80.6 |
| 4 | 35.4 | 89.7 | 34.8 | 93.0 |
| 8 | 61.5 | 98.3 | 59.0 | 98.7 |
| 16 | 80.7 | 103.1 | 78.0 | 101.6 |
| 24 | 89 | 105.2 | 85.7 | 104.2 |

As seen from the results, the release of Effexor 37.5 mg is about 22- 66% faster in 0.1 N HCl and in pH 4.5 with 40%EtOH than it is in pH 1.5 and 4.5 without EtOH

### EXAMPLE 23C

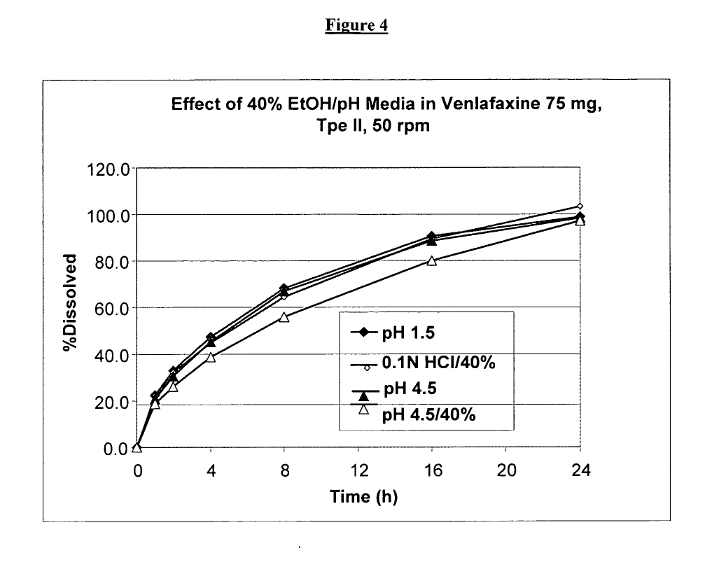

The formulation of Example 22B was subjected to in-vitro dissolution testing with the followin parameters: Apparatus—USP Type II; Volume—900 ml; Rotational Speed—50 rpm; Media—40% Ethanol in 0.1 N HCl or pH 4.5. The results are set forth in Table 14 and FIG. 4.

TABLE 14

| Time | | 0.1N HCl/ | | pH 4.5/ |
|---|---|---|---|---|
| (h) | pH 1.5 | 40% EtOH | pH 4.5 | 40% EtOH |
| 0 | 0.0 | 0 | 0.0 | 0 |
| 1 | 22.5 | 21.6 | 20.7 | 18.9 |
| 2 | 33.2 | 32.2 | 30.5 | 26.1 |
| 4 | 47.5 | 44.9 | 45.5 | 38.8 |
| 8 | 68.2 | 64.3 | 67.0 | 56.1 |
| 16 | 90.7 | 89.2 | 88.7 | 80.2 |
| 24 | 98.7 | 103.3 | 98.2 | 97.2 |

As seen from the results, there is no significant difference in the release of Example 22B in pH 1.5 and in 0.1 N HCl/40%EtOH and the release of the drug is about 4-9% slower in pH 4.5/40%EtOH than it is without EtOH.

### EXAMPLE 23D

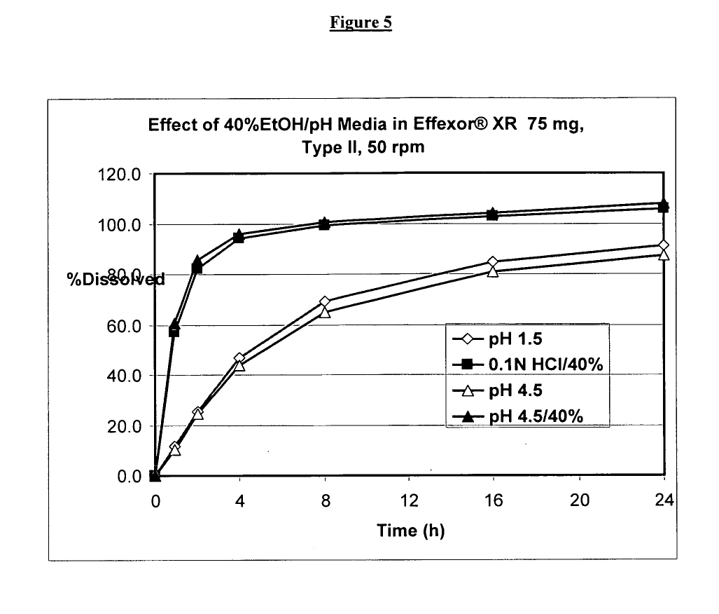

Effexor® XR 75 mg Capsules was subjected to in-vitro dissolution testing with the following parameters: Apparatus—USP Type II; Volume—900 ml; Rotational Speed—50 rpm; Media—40% Ethanol in 0.1 N HCl or pH 4.5. The results are set forth in Table 15 and FIG. 5

TABLE 15

| Time | | 0.1N HCl/ | | pH 4.5/ |
|---|---|---|---|---|
| (h) | pH 1.5 | 40% EtOH | pH 4.5 | 40% EtOH |
| 0 | 0.0 | 0.0 | 0.0 | 0.0 |
| 1 | 11.7 | 57.4 | 10.3 | 60.8 |
| 2 | 25.5 | 82.2 | 24.5 | 85.4 |
| 4 | 47.0 | 94.2 | 44.0 | 96.1 |
| 8 | 69.1 | 99.5 | 65.1 | 100.7 |
| 16 | 84.8 | 102.7 | 80.8 | 104.3 |
| 24 | 91.1 | 105.8 | 87.4 | 107.9 |

As seen from the results, the release of Effexor 75 mg is about 20 - 60% faster in 0.1 N HCl and in pH 4.5 with 40%EtOH than it is in pH 1.5 and 4.5 without EtOH

### EXAMPLE 23E

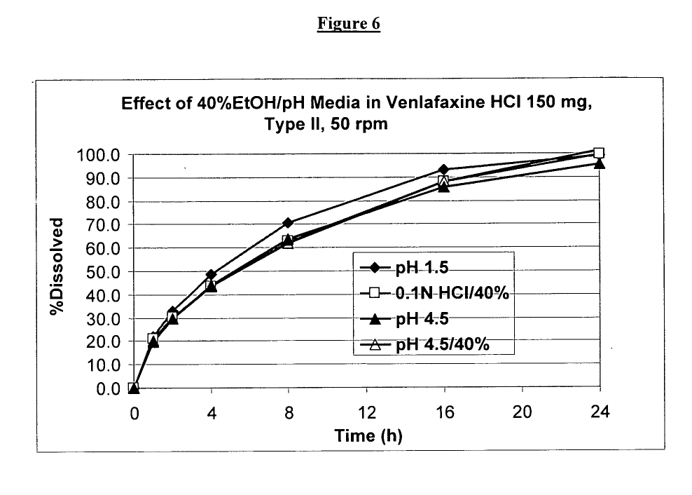

The formulation of Example 22C was subjected to in-vitro dissolution testing with the following parameters: Apparatus—USP Type II; Volume—900 ml; Rotational Speed—50 rpm; Media—40% Ethanol in 0.1 N HCl or pH 4.5. The results are set forth in Table 16 and FIG. 6.

TABLE 16

| Time | | | | |
|---|---|---|---|---|
| (h) | pH 1.5 | 0.1N HCl/40% EtOH | pH 4.5 | pH 4.5/40% EtOH |
| 0 | 0.0 | 0.0 | 0.0 | 0.0 |
| 1 | 21.8 | 21.3 | 19.9 | 20.4 |
| 2 | 32.9 | 30.3 | 29.8 | 29.9 |
| 4 | 48.8 | 43.6 | 43.9 | 43.8 |
| 8 | 70.7 | 63.0 | 63.7 | 62.1 |
| 16 | 93.1 | 88.2 | 85.9 | 87.9 |
| 24 | 99.1 | 99.7 | 95.5 | 101.3 |

As seen from the results, there is no significant difference in the release of Example 22C in pH 4.5 with and without 40%EtOH; and the release of the drug is about 2-7% slower in 0.1 N HCl/40%EtOH than it is in pH 1.5

### EXAMPLE 23F

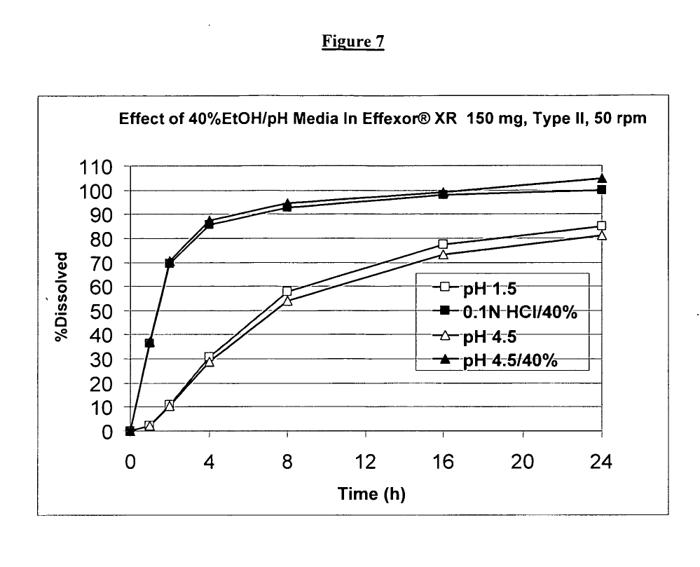

Effexor® XR 150 mg Capsules was subjected to in-vitro dissolution testing with the following parameters: Apparatus—USP Type II; Volume—900 ml; Rotational Speed—50 rpm; Media—40% Ethanol in 0.1 N HCl or pH 4.5. The results are set forth in Table 17 and FIG. 7.

TABLE 17

| Time | | | | |
|---|---|---|---|---|
| (h) | pH 1.5 | 0.1N HCl/40% EtOH | pH 4.5 | pH 4.5/40% EtOH |
| 0 | 0 | 0 | 0 | 0 |
| 1 | 2.4 | 36.5 | 2.4 | 37.1 |
| 2 | 10.8 | 69.6 | 10.5 | 70.4 |
| 4 | 30.8 | 85.7 | 28.9 | 87.4 |
| 8 | 57.7 | 92.9 | 54.2 | 94.6 |
| 16 | 77.3 | 98.0 | 73.2 | 99.3 |
| 24 | 84.9 | 99.8 | 81.2 | 104.7 |

As seen from the results, the release of Effexor 150 mg is about 15 -60% faster in 0.1 N HCl and in pH 4.5 with 40%EtOH than it is in the similar media without the EtOH.

### EXAMPLE 23G

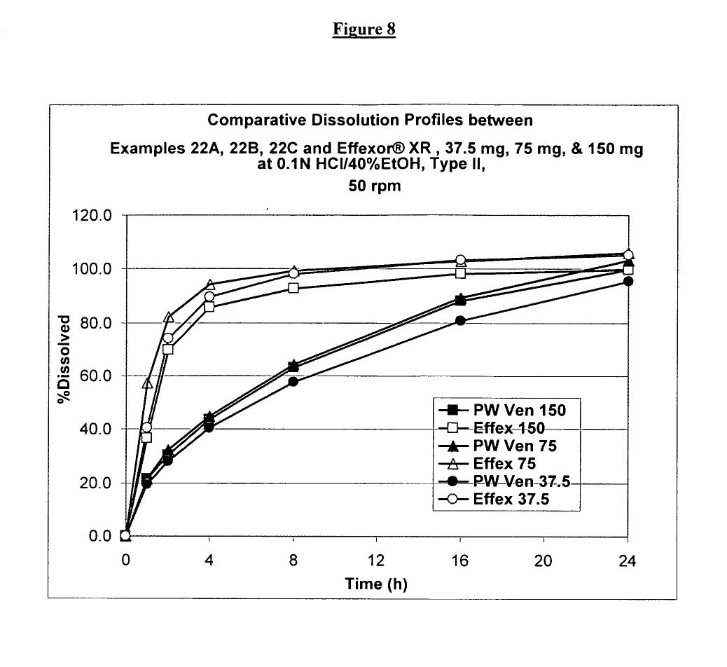

Example 23G compares the results of Examples 23A-F with the following parameters: Apparatus—USP Type II; Volume—900 ml; Rotational Speed—50 rpm; Media—40% Ethanol in 0.1 N HCl. The results are set forth in Table 18 and FIG. 8.

TABLE 18

| Time | | | | | | |
|---|---|---|---|---|---|---|
| (h) | Ex. 22C | Effex 150 | Ex. 22B | Effex 75 | Ex. 22A | Effex 37.5 |
| 0 | 0.0 | 0 | 0 | 0.0 | 0.0 | 0.0 |
| 1 | 21.3 | 36.5 | 21.6 | 57.4 | 19.6 | 40.7 |
| 2 | 30.3 | 69.6 | 32.2 | 82.2 | 28.0 | 73.9 |
| 4 | 43.6 | 85.7 | 44.9 | 94.2 | 40.4 | 89.7 |
| 8 | 63.0 | 92.9 | 64.3 | 99.5 | 57.6 | 98.3 |
| 16 | 88.2 | 98.0 | 89.2 | 102.7 | 80.6 | 103.1 |
| 24 | 99.7 | 99.8 | 103.3 | 105.8 | 95.5 | 105.2 |

### EXAMPLE 23H

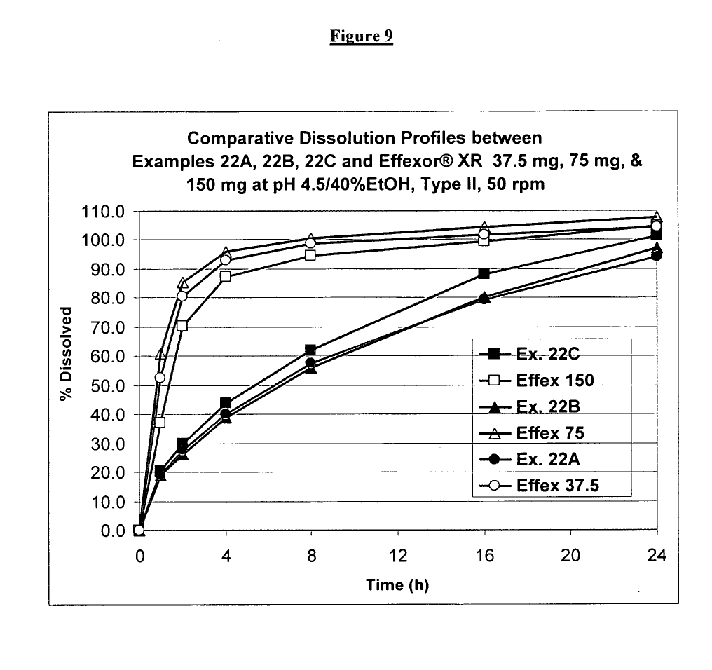

Example 23H compares the results of Examples 23A-F with the following parameters: Apparatus—USP Type II; Volume—900 ml; Rotational Speed—50 rpm; Media—40% Ethanol in pH 4.5. The results are set forth in Table 19 and FIG. 9.

TABLE 19

| Time | | | | | | |
|---|---|---|---|---|---|---|
| (h) | Ex. 22C | Effex 150 | Ex. 22B | Effex 75 | Ex. 22A | Effex 37.5 |
| 0 | 0.0 | 0 | 0 | 0.0 | 0.0 | 0.0 |
| 1 | 20.4 | 37.1 | 18.9 | 60.8 | 18.8 | 52.4 |
| 2 | 29.9 | 70.4 | 26.1 | 85.4 | 27.6 | 80.6 |
| 4 | 43.8 | 87.4 | 38.8 | 96.1 | 40.2 | 93.0 |
| 8 | 62.1 | 94.6 | 56.1 | 100.7 | 57.6 | 98.7 |
| 16 | 87.9 | 99.3 | 80.2 | 104.3 | 79.3 | 101.6 |
| 24 | 101.3 | 104.7 | 97.2 | 107.9 | 94.0 | 104.2 |

As seen in Examples 23A-H, there is no significant effect on the release of the Examples of the present invention in the pH media with of 40%EtOH, but the release of Effexor® XR is dramatically affected in the pH media with 40%EtOH.

1. A controlled release oral dosage form comprising:
a therapeutically effective amount of venlafaxine or a pharmaceutically acceptable salt thereof and a controlled release material;
wherein the amount of venlafaxine or pharmaceutically acceptable salt thereof released at 1 hour in 900 mL 0.1 N HCl (pH 1.5) with 40% EtOH using USP Apparatus II at 50 rpm is within 25% of the amount of venlafaxine or pharmaceutically acceptable salt thereof released at 1 hour in 900 mL 0.1 N HCl (pH 1.5) using USP Apparatus II at 50 rpm.

2. The controlled release dosage form of claim 1, wherein the amount of venlafaxine or pharmaceutically acceptable salt thereof released at 4 hours in 900 mL 0.1 N HCl (pH 1.5) with 40% EtOH using USP Apparatus II at 50 rpm is within 25% of the amount of venlafaxine or pharmaceutically acceptable salt thereof released at 4 hours in 900 mL 0.1 N HCl (pH 1.5) using USP Apparatus II at 50 rpm.

3-7. (canceled)

8. The controlled release dosage form of claim 1, wherein the mean Tmax of venlafaxine is from about 4 hours to about 8 hours after single dose administration.

9-10. (canceled)

11. The controlled release dosage form of claim 1, wherein the mean Tmax of O-desmethyl-venlafaxine is from about 6 hours to about 12 hours after single dose administration.

12-13. (canceled)

14. The controlled release dosage form of claim 1, wherein the mean Cmax of venlafaxine is from about 100 ng/mL to about 200 ng/mL, based on administration of an amount equivalent to about 150 mg venlafaxine base.

15-16. (canceled)

17. The controlled release dosage form of claim 1, wherein the mean Cmax of O-desmethyl-venlafaxine is from about 200 ng/mL to about 350 ng/mL, based on administration of an amount equivalent to about 150 mg venlafaxine base.

18-20. (canceled)

21. The controlled release dosage form of claim 1, which provides 24 hour therapeutic plasma levels of venlafaxine after oral administration to human patients.

22. The controlled release dosage form of any of claims 1-7, comprising a matrix comprising the venlafaxine or pharmaceutically acceptable salt thereof dispersed in the controlled release material.

23. The controlled release dosage form of claim 22, wherein the controlled release material is a gelling agent.

24. The controlled release dosage form of claim 23, wherein said gelling agent comprises a polysaccharide.

25. The controlled release dosage form of claim 24, wherein said polysaccharide is a heteropolysaccharide gum.

26. The controlled release dosage form of claim 25, wherein said gelling agent further comprises a homopolysaccharide gum capable of cross-linking the heteropolysaccharide gum when exposed to an environmental fluid.

27. The controlled release oral dosage form of claim 26, wherein said heteropolysaccharide gum is xanthan gum.

28. The controlled release oral dosage form of claim 26, wherein said homopolysaccharide gum is locust bean gum.

29. The controlled release oral dosage form of claim 23, wherein said matrix further comprises a hydrophobic material.

30. The controlled release oral dosage form of claim 29, wherein said hydrophobic material is selected from the group consisting of a hydrophobic polymer, a cellulosic material, an acrylic polymer, a methacrylic acid polymer, a methacrylic copolymer, hydrogenated vegetable oils, zein, and mixtures thereof.

31. The controlled release oral dosage form of claim 30, wherein said hydrophobic material comprises ethylcellulose.

32. The controlled release oral dosage form claim 23, further comprising an ionizable gel strength enhancing agent capable of crosslinking with said gelling agent and increasing the gel strength when the dosage form is exposed to an environmental fluid.

33. The controlled release oral dosage form of claim 32, wherein said ionizable gel strength enhancing agent comprises an alkali metal or an alkaline earth metal sulfate, chloride, borate, bromide, citrate, acetate, or lactate.

34. The controlled release oral dosage form of claim 32, wherein said ionizable gel strength enhancing agent is selected from the group consisting of calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, sodium fluoride, and mixtures thereof.

35. The controlled release oral dosage form of claim 32, wherein said ionizable gel strength enhancing agent comprises calcium sulfate.

36. The controlled release dosage form of claim 23, wherein said matrix further comprises an inert pharmaceutical diluent.

37. The controlled release dosage form of claim 36, wherein said inert diluent is selected from the group consisting of a monosaccharide, a disaccharide, a polyhydric alcohol, and mixtures thereof.

38. The controlled release dosage form of claim 37, wherein said inert diluent comprises mannitol.

39. The controlled release dosage form of claim 36, wherein the ratio of said inert diluent to said gelling agent is from about 1:5 to about 5:1.

40. The controlled release dosage form of any of claim 23, wherein said matrix further comprises from about 1 to about 20% by weight microcrystalline cellulose.

41. The controlled release dosage form of any of claim 23, wherein said matrix further comprises from about 1 to about 20% by weight silicified microcrystalline cellulose.

42-44. (canceled)

45. The controlled release dosage form of any of claims 1-7, comprising venlafaxine hydrochloride.

46. The controlled release dosage form of any of claims 1-7, comprising venlafaxine hydrochloride in an amount equivalent to about 37.5 mg venlafaxine base.

47-55. (canceled)

56. A controlled release oral dosage form comprising:
a therapeutically effective amount of venlafaxine or a pharmaceutically acceptable salt thereof and a controlled release material;
wherein the amount of venlafaxine or pharmaceutically acceptable salt thereof released at 1 hour in 900 mL 0.1 N HCl (pH 1.5) with 40% EtOH using USP Apparatus II at 50 rpm is less than the amount of venlafaxine or pharmaceutically acceptable salt thereof released at 1 hour in 900 mL 0.1 N HCl (pH 1.5) using USP Apparatus II at 50 rpm.

57-69. (canceled)
